(19) United States
(12) Patent Application Publication
(10) Pub. No.: US 2024/0158386 A1
(43) Pub. Date: May 16, 2024

(54) BENZOXAZOLONE INHIBITORS OF INFLAMMASOMES

(71) Applicants: ICAHN SCHOOL OF MEDICINE AT MOUNT SINAI, New York, NY (US); THE UNITED STATES GOVERNMENT AS REPRESENTED BY THE DEPARTMENT OF VETERANS AFFAIRS, Washington, DC (US)

(72) Inventors: Robert DEVITA, New York, NY (US); Giulio M. PASINETTI, New York, NY (US); Maria SEBASTIAN-VALVERDE, New York, NY (US); Francis HERMAN, New York, NY (US); Kunal KUMAR, New York, NY (US)

(73) Assignees: ICAHN SCHOOL OF MEDICINE AT MOUNT SINAI, New York, NY (US); THE UNITED STATES GOVERNMENT AS REPRESENTED BY THE DEPARTMENT OF VETERANS AFFAIRS, Washington, DC (US)

(21) Appl. No.: 18/548,348

(22) PCT Filed: Mar. 1, 2022
(86) PCT No.: PCT/US2022/070881

§ 371 Date: Aug. 30, 2023

Related U.S. Application Data
(60) Provisional application No. 63/155,372, filed on Mar. 2, 2021.

(51) Int. Cl. C07D 413/14; A61P 29/00; C07D 413/12

(52) U.S. Cl. CPC C07D 413/14; A61P 29/00; C07D 413/12

(57) ABSTRACT

Compounds which are benzoxazolone derivatives are disclosed, including compounds of the following genus:

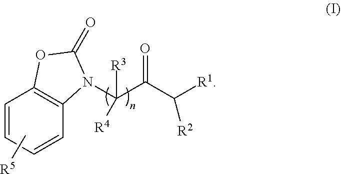

The compounds possess anti-inflammasome properties and exhibit anti-fibrotic and anti-proliferative effects. They are useful in inhibiting the activation of NLRP3 or NLRC4 receptors, and in the treatment of a variety of neuroinflammatory disorders such as autoimmune diseases, type-2 diabetes, Cryopyrin-Associated Autoinflammatory Syndromes, Alzheimer's disease, Parkinson's disease, Amyotrophic Lateral Sclerosis, Multiple Sclerosis, and rheumatoid arthritis.

### TECHNICAL FIELD

This invention relates to benzoxazolone-containing compounds that are inhibitors of Nucleotide Binding Oligomerization Domain like Receptors (NLR) inflammasomes. The compounds disclosed are useful in the treatment of various neuroinflammatory diseases, such as autoimmune diseases, type-2 diabetes, or neurodegenerative disorders.

### BACKGROUND OF THE INVENTION

Nucleotide Binding Oligomerization Domain like Receptors (NLR) is a group of cytoplasmic pattern-recognition receptors that mediate the initial innate immune response to microbial infections, stress, and host-derived factors. Upon activation, some NLRs form inflammasomes, which through recruitment and activation of caspase-1, promote the proteolytic cleavage, maturation, and secretion of the proinflammatory cytokines IL-1β and IL-18. Additionally, activated caspase-1 cleavages Gasdermin-D, triggering a non-apoptotic type of cell death called pyroptosis, which mediates the secretion of the mature cytokines to the cytoplasm. Among the NLRs, the NOD-like receptor family pyrin domain containing 3 (NLRP3) is so far the best studied and characterized one. Nevertheless, another inflammasome, the NLR family CARD domain containing 4 (NLRC4), is progressively collecting more attention. Structurally, both inflammasomes present a Leucine-Rich Repeat domain (LRR) and a nucleotide-binding and oligomerization domain (NACHT) domain. Additionally, NLRP3 has a pyrin domain that interacts with the ASC adaptor protein, which recruits caspase-1. By its part, NLRC4 directly interacts with caspase-1 through a card domain. However, upon NLRP3 and NLRC4 activation, apoptosis-associated speck-like protein containing a CARD (ASC) homo-oligomerizes forming large specks that propagate inflammation. Interestingly, the NACHT domain of both proteins holds an ATPase activity that is essential for inflammasome activation , which makes this domain to appear as an appealing therapeutic target.

NLRP3 and NLRC4 have a primary role in mediating the first line of host defense against microbial pathogens, but their prolonged activation leads to a broad spectrum of diverse diseases. For instance, gain of function mutations in the NLRP3 NACHT domain cause different forms of Cryopyrin-Associated Autoinflammatory Syndromes (CAPSs), while genetic alterations in the NLRC4 gene produce autoinflammatory syndromes. Moreover, NLRP3 and NLRC4 are indirectly involved in several conditions in which the prolonged exposure to stress or danger signals induces over-reactivity of the inflammasome. Under these situations, Danger Associated Molecular Patterns (DAMPS)—such as the alarmin HMGB 1—trigger inflammasome activation in a process known as sterile inflammation. Sterile inflammation has an especially pernicious impact in a batch of neurological disorders such as Alzheimer's disease (AD), Parkinson's disease (PD), Amyotrophic Lateral Sclerosis (ALS), or Multiple Sclerosis (MS), where inflammatory cytokines produced by microglia and astrocytes establish a feedback loop that recruits more inflammatory cells, enhancing the inflammation process. In this scene, the role of the ASC adaptor protein in AD deserves special mention. As aforementioned, inflammasome activation triggers the aggregation of ASC into large fibrils forming specks, which are released to the extracellular medium, where they recruit more inflammatory cells, amplifying inflammation. Also, ASC specks recruit amyloid-β, promoting its oligomerization and aggregation acting as an inflammation-driven cross-seed for amyloid-β pathology, and worsen the pathology of AD.

The average survival from onset of Amyotrophic Lateral Sclerosis (ALS) to death is 2-4 years. No cure for ALS is known and the available treatments only delay symptoms and elicit modest benefits in survival. Therefore, there is an urgent need for new, more effective therapeutics targeting the underlying disease mechanisms. Recent investigations find that dysregulated innate immune activity may act as a pathogenic mechanism that affects neuronal structure and function. In particular, inflammasome complexes, which translate pathogenic and sterile stress signals into inflammatory responses, may represent one dysregulated immune pathway in ALS given their pathogenic effects in other neurodegenerative disorders.

NLRP3 is unique in its sensitivity to recognize oxidative stress and initiate inflammation, and may represent a novel therapeutic target for ALS given the critical role of reactive oxygen species (ROS) in ALS. In response to cellular exposure to external stress signals such as ROS, NLRP3 oligomerizes into a heteromeric complex containing Apoptosis-associated speck-like protein containing a CARD (ASC) and pro-caspase-1 known as the NLRP3 inflammasome, resulting in cleavage of pro-inflammatory cytokines such as interleukin 1β (IL-1β) and IL-18 and their secretion to induce neuroinflammatory responses. As increased NLRP3 inflammasome activity has been reported in other neurodegenerative disorders including Alzheimer's disease (AD) and Parkinson's disease (PD), NLRP3 activation may act as a pathophysiological effector across ALS phenotypes, and NLRP3 inhibitors represent a powerful therapeutic approach for familial and sporadic ALS forms. It has been reported that select phenolic metabolites derived from a polyphenol-rich grape seed extract (GSE) interfere with the assembly of (β-amyloid (Aβ) aggregates. As shown in in vitro preliminary studies, one polyphenolic metabolite, 3-hydroxybenzoic acid (3-HBA) is capable of inhibiting NLRP3-dependent inflammatory responses and exhibited promising in vivo evidence of preventing ALS-related pathophysiology and downstream deficits in behavior and cognitive function. Other small molecule compounds displayed in vitro efficacy in inhibiting NLRP3-specific inflammatory responses.

Currently, most of the therapeutic efforts to treat inflammasome-related pathologies focus on the specific inhibition of NLRP3. Thus, there are a plethora of small molecule inhibitors for NLRP3. For instance, and among many others; glyburide presents in-vitro anti-NLRP3 activity, but a limited effect in-vivo; β-hydroxybutyrate inhibits NLRP3 activation by disrupting NLRP3-ASC oligomerization; and MCC950 is a very potent and specific NLRP3 inhibitor, although its action mechanism is still to be clarified. Although this strategy is highly effective against CAPSs, for the treatment of neurological disorders in which different inflammasomes are involved, a less specific approach appears as a better tactic. Following this latter approach, a few agonists of the IL-1β receptor have been successfully used in patients with CAPS and rheumatoid arthritis, but these drugs fail to block pathologies driven by caspase-1, or ASC oligomers. Consequently, there is a compelling need to develop new “broad-spectrum” inflammasome inhibitors.

### SUMMARY OF THE INVENTION

The present invention provides, in a first aspect, a compound of formula I:

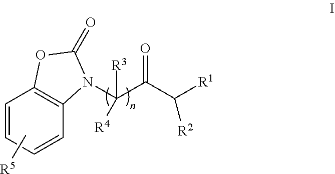

wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

The present invention provides, in a second aspect, pharmaceutical compositions comprising the compounds described herein.

In a third aspect, the invention relates to methods and uses of compounds or pharmaceutical compositions described herein for the treatment of a neuroinflammatory disorder.

In a fourth aspect, the invention relates to methods and uses of compounds or pharmaceutical compositions described herein for the treatment of a neuroinflammatory disorder involving the dysregulation of one or more NLRs.

In a fifth aspect, the invention relates to methods and uses of compounds or pharmaceutical compositions described herein for inhibiting the formation of inflammasomes.

In a sixth aspect, the invention relates to methods and uses of compounds or pharmaceutical compositions described herein for inhibiting the activation of NLRP3 or NLRC4 receptors.

These, and other objects, features and advantages of this invention will become apparent from the following detailed description of the various aspects of the invention taken in conjunction with the accompanying drawings.

### BRIEF DESCRIPTION OF THE DRAWINGS

FIG. 1A depicts the structures of Compounds C75, C78, and C97.

FIG. 1B depicts the dose-response curves for Compounds C75, C78, and C97 for NLRP-mediated IL-1β.

FIG. 1C depicts the inhibitory effect of Compounds C75, C78, and C97 on NLRC4-mediated IL-1β production.

FIG. 1D depicts the effect of Compounds C75, C78, and C97 on the activity of the AIM2 inflammasome.

FIG. 1E depicts the inhibition of ASC specks formation by different concentrations of Compounds C75, C78, and C97.

FIG. 1F depicts the antipyroptotic effect of Compounds C75, C78, and C97.

FIG. 2A shows the structure of the common core identified as the pharmacophore (in square) and chemical structures of the “R” substituents of the selected benzoxazolone acetamide analogs.

FIG. 2B shows the tnhibitory effect of various benzoxazolone acetamide analogs in NLRP3-mediated IL-1β production.

FIG. 2C shows the inhibition of NLRP3 activation by different concentrations of selected benzoxazolone acetamide analogs.

FIG. 2D shows the structure and molecular formula of C77.

FIG. 2E shows the cytotoxicity exerted by different concentrations of C77 in microglial murine cultures.

FIG. 3A shows the dose-dependent inhibition of NLRP3 and NLRC4.

FIG. 3B shows the effect of C77 on NLRP3, AIM-2, NLRP7, IL-6, and TNF-α.

FIG. 3C shows the effect of C77 on NLRP3, caspase-1, IL-1β, IL-6, IL-8, and TNF-α gene expression.

FIG. 3D shows the effect of C77 concentration on ATPase activity.

FIG. 3E shows the fluorescence microscopic images for ASC specks formation following NLRP3 activation.

FIG. 3F shows C77 inhibition of ACS oligomerization.

FIG. 4A shows the scheme of the experimental procedure to illustrate the in-vivo effect of C77.

FIG. 4B shows the in-vivo effect of C77 on IL-18 production in the frontal cortex of mice.

FIG. 4C shows IL-1β quantification in different brain regions of mice.

FIG. 4D shows RNA expression levels of inflammatory genes in the brains of mice.

FIG. 5A depicts the HPLC chromatogram for Compound C77.

FIG. 5B depicts the HPLC chromatogram for Compound C78.

### DETAILED DESCRIPTION OF THE INVENTION

Proposed herein is a new therapeutic approach to treat neuroinflammatory disorders, consisting of targeting the highly conserved ATPase activity of the NACHT domain of NLR proteins.

Inflammasomes are macromolecular complexes involved in the host response to external and endogenous danger signals. Inflammasome-mediated sterile inflammation plays a central role in several human conditions such as autoimmune diseases, type-2 diabetes, or neurodegenerative disorders. Among all inflammasomes, NLRP3 is the best-characterized one, which has brought great efforts to find specific inhibitors. Additionally, NLRC4 has recently attracted considerable attention due to recent evidence on its implication in different neuroinflammatory pathologies. Here, a twofold therapeutic approach to inhibit both inflammasomes for treating inflammation-related neurological disorders is proposed. This methodology develops inflammasome inhibitors by targeting the ATPase activity of the Nucleotide-Binding domain. Compounds have been found that are small molecules with anti-inflammasome properties.

Substituents are generally defined when introduced and retain that definition throughout the specification and in all independent claims.

In a composition aspect, the invention relates to compounds of formula I:

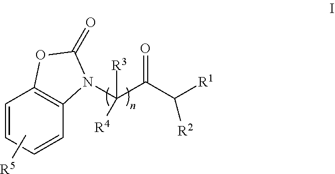

wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
RC is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

R^{a} and R^{b} are independently selected in each instance from hydrogen, (C₁-C₈)hydrocarbon, —OH, and optionally substituted heterocycle. To be clear, since R¹ is —(CR^{a}R^{b})ₚ-Q-R¹⁰, if p is 2, then each R^{a} and each R^{b} is selected from hydrogen, (C₁-C₈)hydrocarbon, —OH, and optionally substituted heterocycle. Each R^{a} and each R^{b} may be the same or different. As a non-limiting example when p is 2, the —(CR^{a}R^{b})ₚ moiety could be —CH₂—C(—OH)(furan)-, wherein the first R^{a} is hydrogen, the first R^{b} is hydrogen, the second R^{a} is —OH, and the second R^{b} is furan. In another non-limiting example when p is 2, the —(CR^{a}R^{b})ₚ moiety may be —(CH₂)₂—.

### ABBREVIATIONS AND DEFINITIONS

Unless defined otherwise, all technical and scientific terms used herein have the same meaning as is commonly understood by one of ordinary skill in the art to which this disclosure belongs. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference. In the event that there is a plurality of definitions for terms cited herein, those in this section prevail unless otherwise stated.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

A “patient” or “subject,” as used herein, includes both humans and other animals, particularly mammals. Thus, the methods are applicable to both human therapy and veterinary applications. In some embodiments, the patient is a mammal, for example, a primate. In some embodiments, the patient is a human.

Treatment can involve administering a compound described herein to a patient diagnosed with a disease and may involve administering the compound to a patient who does not have active symptoms. Conversely, treatment may involve administering the compositions to a patient at risk of developing a particular disease, or to a patient reporting one or more of the physiological symptoms of a disease, even though a diagnosis of this disease may not have been made.

The terms “administer”, “administering” or “administration” in reference to a dosage form of the invention refers to the act of introducing the dosage form into the system of subject in need of treatment. When a dosage form of the invention is given in combination with one or more other active agents (in their respective dosage forms), “administration” and its variants are each understood to include concurrent and/or sequential introduction of the dosage form and the other active agents. Administration of any of the described dosage forms includes parallel administration, co-administration or sequential administration. In some situations, the therapies are administered at approximately the same time, e.g., within about a few seconds to a few hours of one another.

A “therapeutically effective” amount of a compound described herein is typically one which is sufficient to achieve the desired effect and may vary according to the nature and severity of the disease condition, and the potency of the compound. It will be appreciated that different concentrations may be employed for prophylaxis than for treatment of an active disease. A therapeutic benefit is achieved with the amelioration of one or more of the physiological symptoms associated with the underlying disorder such that an improvement is observed in the patient, notwithstanding that the patient may still be afflicted with the underlying disorder.

Throughout this specification the terms and substituents retain their definitions.

Hydrocarbon refers to any substituent comprised of hydrogen and carbon as the only elemental constituents. (C₁-C₈)hydrocarbon includes alkyl, cycloalkyl, polycycloalkyl, alkenyl, alkynyl, aryl and combinations thereof. Non-limiting examples of hydrocarbons include methyl, benzyl, phenethyl, cyclohexylmethyl, and naphthylethyl.

Unless otherwise specified, alkyl (or alkylene) is intended to include linear, branched, or cyclic saturated hydrocarbon structures and combinations thereof. A combination would be, for example, cyclopropylmethyl. Unless otherwise specified, alkyl refers to alkyl groups from 1 to 20 carbon atoms, in some instances 1 to 10 carbon atoms, in some instances 1 to 6 carbon atoms, in some instances 1 to 4 carbon atoms, and in some instances 1 to 3 carbon atoms. Examples of alkyl groups include methyl, ethyl, propyl, isopropyl, cyclopropyl, n-butyl, s-butyl, t-butyl and the like. Cycloalkyl is a subset of alkyl and includes cyclic hydrocarbon groups of from 3 to 8 carbon atoms and, in some instances, from 3 to 6 carbon atoms. Examples of cycloalkyl groups include c-propyl, c-butyl, c-pentyl, norbornyl and the like.

Unless otherwise specified, the term “carbocycle” is intended to include ring systems in which the ring atoms are all carbon but of any oxidation state. Thus (C₃-C₁₀) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Heterocycle means a cycloalkyl or aryl carbocycle residue in which from one to four carbons is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic or aromatic. Examples of heterocycles that fall within the scope of the invention include pyrrolidine, piperidine, piperazine, azepane, azepine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl. In some embodiments, a monocyclic heterocycle contains four, five, six, or seven members.

Heteroaryl is a subset of heterocycle in which the heterocycle is aromatic. In some instances, the heteroaryl contains five or six ring members. Examples of these include isoxazole, oxazole, thiazole, furan, pyrazole, thiophene, thiazole, pyrrole, imidazole, isothiazole, oxadiazole, triazole, thiadiazole, pyridine, pyridazine, pyrimidine, pyrazine, and triazine, and the like. In some instances, heteroaryl may include isoxazole, oxazole, imidazole, and pyrazole.

Alkoxy or alkoxyl refers to groups of from 1 to 20 carbon atoms, preferably 1 to 10 carbon atoms, more preferably 1 to 6 carbon atoms of a straight or branched configuration attached to the parent structure through an oxygen. Examples include methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

The term “halogen” means fluorine, chlorine, bromine or iodine atoms. In one embodiment, halogen may be a fluorine or chlorine atom.

The terms “haloalkyl” and “haloalkoxy” mean alkyl or alkoxy, respectively, substituted with one or more halogen atoms.

As used herein, the term “optionally substituted” may be used interchangeably with “unsubstituted or substituted”. The term “substituted” refers to the replacement of one or more hydrogen atoms in a specified group with a specified radical. For example, unless otherwise specified, substituted alkyl, aryl, cycloalkyl, heterocyclyl, etc. refer to alkyl, aryl, cycloalkyl, or heterocyclyl wherein one or more H atoms in each residue are replaced with halogen, haloalkyl, alkyl, acyl, alkoxyalkyl, hydroxy lower alkyl, carbonyl, phenyl, heteroaryl, benzenesulfonyl, hydroxy, lower alkoxy, haloalkoxy, oxaalkyl, carboxy, alkoxycarbonyl [—C(═O)O-alkyl], alkoxycarbonylamino [HNC(═O)O-alkyl], aminocarbonyl (also known as carboxamido) [—C(═O)NH₂], alkylaminocarbonyl [—C(═O)NH-alkyl], cyano, acetoxy, nitro, amino, alkylamino, dialkylamino, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, sulfoxide, sulfone, sulfonylamino, alkylsulfinyl, alkylsulfonyl, acylaminoalkyl, acylaminoalkoxy, acylamino, amidino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, benzyloxyphenyl, and benzyloxy. “Oxo” is also included among the substituents referred to in “optionally substituted”; it will be appreciated by persons of skill in the art that, because oxo is a divalent radical, there are circumstances in which it will not be appropriate as a substituent (e.g. on phenyl). In one embodiment, 1, 2, or 3 hydrogen atoms are replaced with a specified radical. In the case of alkyl and cycloalkyl, more than three hydrogen atoms can be replaced by fluorine; indeed, all available hydrogen atoms could be replaced by fluorine. In some embodiments, substituents are halogen, haloalkyl, alkyl, acyl, hydroxyalkyl, hydroxy, alkoxy, haloalkoxy, aminocarbonyl oxaalkyl, carboxy, cyano, acetoxy, nitro, amino, alkylamino, dialkylamino, alkylthio, alkylsulfinyl, alkyl sulfonyl, alkylsulfonylamino aryl sulfonyl, arylsulfonylamino, and benzyloxy.

The compounds described herein contain three or more asymmetric centers and may thus give rise to enantiomers, diastereomers, and other stereoisomeric forms which may be defined in terms of absolute stereochemistry as (R)- or (S)-. The present invention is meant to include all such possible diastereomers as well as their racemic and optically pure forms. Optically active (R)- and (S)-isomers may be prepared using homo-chiral synthons or homo-chiral reagents, or optically resolved using conventional techniques. When the compounds described herein contain olefinic double bonds or other centers of geometric asymmetry, and unless specified otherwise, it is intended to include both (F)- and (Z)-geometric isomers. Likewise, all tautomeric forms are intended to be included.

The graphic representations of racemic, ambiscalemic and scalemic or enantiomerically pure compounds used herein are a modified version of the denotations taken from Maehr J. Chem. Ed. 62, 114-120 (1985): simple lines provide no information about stereochemistry and convey only connectivity; solid and broken wedges are used to denote the absolute configuration of a chiral element; solid and broken bold lines are geometric descriptors indicating the relative configuration shown but not necessarily denoting racemic character; and wedge outlines and dotted or broken lines denote enantiomerically pure compounds of indeterminate absolute configuration. For example, the graphic representation

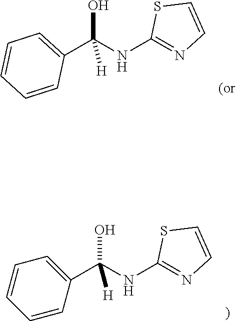 [SMILES: O[C@H](Nc1nccs1)c1ccccc1 ; O[C@@H](Nc1nccs1)c1ccccc1]

indicates either, or both, of the enantiomers below:

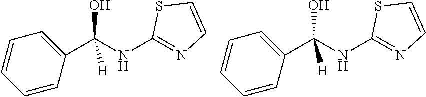

in any ratio, from pure enantiomers to racemates. The graphic representation:

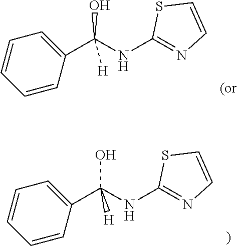 [SMILES: O[C@H](Nc1nccs1)c1ccccc1 ; O[C@@H](Nc1nccs1)c1ccccc1]

indicates a single enantiomer of unknown absolute stereochemistry, i.e. it could be either of the two preceding structures, as a substantially pure single enantiomer.

For the purpose of the present disclosure, a “pure” or “substantially pure” enantiomer is intended to mean that the enantiomer is at least 95% of the configuration shown and 5% or less of other enantiomers. Similarly, a “pure” or “substantially pure” diastereomer is intended to mean that the diastereomer is at least 95% of the relative configuration shown and 5% or less of other diastereomers.

It may be found upon examination that certain species and genera are not patentable to the inventors in this application. In this case, the exclusion of species and genera in applicants' claims are to be considered artifacts of patent prosecution and not reflective of the inventors' concept or description of their invention, which encompasses all members of the genus that are not in the public's possession. As used herein, and as would be understood by the person of skill in the art, the recitation of “a compound” -unless expressly further limited—is intended to include salts of that compound. In a particular embodiment, the term “compound of formula” refers to the compound or a pharmaceutically acceptable salt thereof.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

Also provided herein is a pharmaceutical composition comprising a compound disclosed above, or a pharmaceutically acceptable salt form thereof, and a pharmaceutically acceptable carrier or diluent.

While it may be possible for the compounds of the formulae disclosed herein to be administered as the raw chemical, it is preferable to present them as a pharmaceutical composition. According to a further aspect, the present invention provides a pharmaceutical composition comprising a compound of formula I or a pharmaceutically acceptable salt thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula I or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, and chlorine include ²H, ³H, ¹³C, ¹⁴C, ¹⁵N, ³⁵S, ¹⁸F, and ³⁶Cl, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Compounds that contain isotopes ¹¹C, ¹³N, ¹⁵O and ¹⁸F are well suited for positron emission tomography. Radiolabeled compounds of formula I of this invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

### Materials and Methods

### Cell Culture and Treatment

THP1 human macrophages. THP1 human monocytes were purchased from the American Type Culture Collection (ATCC® TIB-202™) and maintained in RPMI-1640 medium, supplemented with 10% fetal bovine serum, and 2-mercaptoethanol at a final concentration of 0.05 mM. Monocyte differentiation into macrophages was induced upon treatment with 100 ng/ml phorbol 12-myristate 13-acetate (PMA) for 48 hours. Then, cells were washed three times with culture medium without serum and let to rest for 24 hours in the complete culture medium.

THP1 macrophages were preincubated for 1 hour with the 100 hits from the primary screening, at a concentration of 100 μM—control samples were pretreated with same DMSO volume. NLRP3 priming was then induced by the addition of 400 ng/ml LPS. After 3 hours of incubation with LPS, 4.5 mM ATP was added for 45 minutes for inflammasome activation. Supernatants were collected for IL-1β determination and viability assays. 5 μM MCC950 was used as a positive control of NLRPP3 inhibition.

Murine primary microglia. Mixed cortical cultures were prepared as previously described (Bronstein, R. et al. J. Vis. Exp. JoVE 50647 (2013) doi:10.3791/50647). Briefly, cortices from 2-3 days old, WT, or NLRP3 KO pups were isolated, digested, and seeded at a density of 8 cortices per 10 ml petri dish. Every three days, the medium was replaced by fresh culture medium (DMEM, 10% FBS, 1% penicillin-streptomycin). After three weeks, mixed glial cultures had reached confluence, and microglia cells were isolated by mild trypsinization as previously described (Saura, J. et al. Glia 44, 183-189 (2003)). In brief, cells were washed with culture medium without FBS and treated with a mixture of trypsin 0.25% (without EDTA) and DMEM-F12 medium in a 1:3 proportion. After an incubation period of approximately 40 min, an intact layer of mixed glial cells detached, leaving microglia attached firmly to the bottom of the Petri-dish. Pure microglia were then isolated by 15 min incubation with trypsin (0.05%)-EDTA at 37° C., followed by gentle shaking. Cells were counted and seeded at the desired density, typically 7.5·10⁴ cells/well in 24 well-plates.

NLRP3 inflammasome activation assay. Primary microglia cultures were incubated with the compounds that inhibited IL-1β production in THP1 macrophages. Cells pretreated with 100 μM of the compounds—or with the same DMSO volume, in the case of control samples—were primed with 400 ng/ml LPS for 3 hours, and activated with 4.5 mM ATP for 45 minutes or 21.1M nigericin for 1 hour. Dose-response curves were conducted by incubating microglia cultures with different concentrations of the selected compounds, ranging from 0.001 to 100 μM, 1 hour before NLRP3 activation with LPS and ATP. Supernatants were collected for IL-1β and toxicity assessments.

AIM2, NLRP7, and NLRC4 inflammasomes activation assays. Microglia from NLRP3 KO mice were pre-treated with 100 μM C75, C78, C97, or C77, during one hour. AIM2 inflammasome activation was induced through the addition of 100 ng/ml LPS for 2 hours, followed by transfection of 1 μg poly(dA:dT) using Lipofectamine 2000 during 2 hours, following the manufacturer recommendations. NLRC4 was activated by treatment with 150 ng/ml LPS for 2 hours, before transfection of 1 μg flagellin during 2 hours, using lipofectamine 2000 as transfection reagent. Negative and positive controls were pre-treated with the same DMSO percentage as cells treated with the inhibitors. Negative controls were kept untreated after DMSO addition, while LPS-poly(dA:dT) or LPS-flagellin was added to positive controls for AIM2, and NLRC4 activation, respectively. For activation of the NLRP7 inflammasome different conditions were assessed: treatment of WT microglia cultures with 400 ng/ml fibroblast-stimulating lipopeptide-1 (fsl-1) for 7 hours, priming with 150 ng/ml LPS for 1 hour and treatment with 400 ng/ml fsl-1 for 7 hours, and priming with 150 ng/ml LPS and transfection of 400 ng/ml fsl-1 with lipofectamine. MCC950 was used as a negative control for NLRC4 and AIM-2 inhibition. Cells were pretreated with 5 μM MCC950 before LPS addition, and 2 hours later the NLRC4 and AIM-2 activation was induced through addition of their specific activators. Cell supernatants were collected for IL-1β measurement.

m-Cerulean ASC labeled macrophages. NLRP3 Knock Out immortalized murine macrophages, stably expressing m-Cerulean-ASC, and NLRP3-Flag were a kind donation of Prof. Eicke Latz, (Institute of Innate Immunity, University of Bonn, Germany). Murine macrophages were maintained in high glucose DMEM medium supplemented with 10% FBS, 1% sodium pyruvate, and passaged every 2-3 days, before reaching confluence.

For ASC speck formation evaluation, murine macrophages were seeded in 96 well SCREENSTAR black microplates with clear base, 24 hours before stimulation. Then, cells were incubated for 1 hour with different concentrations of the inhibitors, or with the same DMSO volume for positive and negative controls for NLRP3 activation—DMSO-LPS-ATP or only DMSO, respectively. 0.5 μM MCC950 was used as a positive control for inhibition of NLRP3-mediated ASC oligomerization. NLRP3 activation was induced through treatment with 250 ng/ml LPS for 2.5 hours, followed by addition of 4.5 mM ATP for 45 min. Cells were then fixed and counterstained in a single step, as previously described (Stutz, A. et al. ASC Speck Formation as a Readout for Inflammasome Activation. in The Inflammasome (eds. De Nardo, C. M. & Latz, E.) vol. 1040 91-101 (Humana Press, 2013).). For that, a stock solution of DRAQS in formaldehyde was used, being the final concentrations 1% formaldehyde and 2.5 μM DRAQ5.

Cell viability assays. The cytotoxic effect of the compounds identified by in-vitro screening was assayed with the Cyto Tox 96® NonRadioactive Cytotoxicity Assay (Promega), following the recommendations of the manufacturer.

Pyroptosis measurement. The ability of C75, C77, C78, and C97 to inhibit pyroptosis was determined by measuring the LDH released by pyroptotic cells into the culture medium. 350 ng/ml LPS was added to primary microglial cultures pre-treated with different concentrations of the inhibitors. After 3 hours of treatment, pyroptosis was induced through the addition of 4.5 mM ATP, for 45 min. Cell supernatants were collected for LDH assessment, using the Cyto Tox 96® NonRadioactive Cytotoxicity Assay (Promega), following manufacturer the recommendations.

ASC oligomerization determination. The inhibitory effect of the selected compounds on ASC oligomerization was tested using m-Cerulean ASC labeled macrophages. ASC specks in cells pretreated with C75, C77, C78, and C97, before NLRP3 activation (as above indicated), were visualized with a widefield Leica DMi8 fluorescence microscope, using the 20× objective. Cy5 (λ_{exc} 630 nm) and DAPI (λ_{exc} 405 nm) filters were used for m-cerulean and DRAQ5 visualization, respectively. Z-stack images were taken. For image analysis and speck quantification, ImageJ was used https://imagej.nih.gov/ij/index.html. Speck and nuclei quantification was performed as previously described in Stutz et al. When required (overlapping nuclei), the watershed separation algorithm was used to separate the particles. All results were subject to manual validation. The rate of speck positive cells was calculated by normalizing the number of speck positive cells to the number of nuclei. Three biological replicates were taken for each treatment group.

Cytokine quantification. IL-1β in cell supernatants and brain tissue samples—after the different treatment groups—was quantified using the Mouse IL-1 beta/IL-1F2 DuoSet ELISA kit (R&D systems), IL-6 in cell culture supernatant was measured through the Mouse IL-6 ELISA kit (ThermoFisher), TNF-α in cell culture supernatants was determined using the Mouse TNF-alpha DuoSet ELISA (R*D systems), and IL-18 in brain tissue was measured through the IL-18 Mouse ELISA Kit (Invitrogen) following the manufacturers' recommended protocol. IL-1β precursor and cleaved IL-1β in the FC of mice treated with vehicles, LPS, or C77-LPS was assessed by western blot. All samples were run in technical duplicates.

Inhibition of the NLRP3 ATPase activity. The efficacy of C77 to inhibit ATP hydrolysis by NLRP3 was directly assayed using a cell-free assay. Human recombinant NLRP3 protein (BPS Bioscience, San Diego CA) was incubated at 37° C. with different concentrations of C77 for 20 min, as previously described (Cocco, M., et al. (2017) Development of an Acrylate Derivative Targeting the NLRP3 Inflammasome for the Treatment of Inflammatory Bowel Disease. J. Med. Chem. 60, 3656-3671). 10 μM ultrapure ATP was then added, and reaction mixtures were further incubated for 45 min. ATP conversion into ADP was determined using the ADP-Glo Kinase Assay (Promega), following the recommendations of the manufacturer, in a SpectraMax i3x Multimode Detection Platform.

Gene expression analysis. RNA was isolated from frontal cortex from C57BL/6J mice or from THP1 human cultures using the RNeasy Mini Kit (QIAGEN, Hilden, Germany), following the recommendations of the manufacturer. RNA concentration was determined, and cDNA was synthesized using the High-Capacity cDNA Reverse Transcription Kit with RNase Inhibitor (applied biosystems™). The expression levels of inflammatory genes—NLRP3, caspase-1, IL-1β, TNF-α, NLRC4, IL-18, and IL6—were determined in 4 replicates through PowerUp SYBER Green Master Mix (Thermofisher) using an ABI PRISM 70900HT Sequence Detection System. Hypoxanthine phosphoribosyltransferase (HPRT) was used as an internal control in samples from mouse brains, and GAPDH in the case of THP1 lysates. Gene expression levels were normalized to control samples (mice or THP1 cells treated with PBS).

Animals used in in-vivo assessments. WT C57BL/6J mice (stock #000664) were obtained from The Jackson Laboratory. Mice were housed in groups of five animals per cage on a 12:12 hours light/dark cycle. Water and food were provided ad libitum. 8-10 weeks males were used for experiments. All procedures were approved by the Institutional Animal Care and use Committee of the Icahn School of Medicine at Mount Sinai.

In-vivo assessment of C77. The ability of C77 to inhibit NLRP3 in-vivo was evaluated using WT C57BL/6J mice. C77 (50 mg/kg), or the same volume of DMSO vehicle, was intraperitoneally injected 2 hours before of LPS (15 mg/kg) or vehicle (PBS) administration. 1.5 hours later, the animals were sacrificed and their brain was isolated. Cortex, frontal cortex, hippocampus, and cerebellum regions from the right hemisphere were obtained for IL-1β analysis. The left hemisphere was used for gene expression analysis. N=5 animals per group.

Statistical analysis. Unless otherwise indicated, all the experiments included in this manuscript were performed by triplicate. Results are expressed as mean±standard deviation (SD) or as mean±standard error (SE) of the regression. A one-way analysis of variance (ANOVA) followed by Tukey's post-test (confidence interval=95%) was performed using GraphPad Prism 8 software (GraphPad Software, San Diego CA) to determine statistical significance between different treatment groups. Unless otherwise indicated, significance intervals are expressed as * p≤0.05, ¥ p≤0.01, § p≤0.001, ¶ p≤0.0001.

### Results

Compounds C56, C61, C75, C78, C87, and C97 were chosen for further characterization. To discard compounds potentially toxic, the cytotoxicity of the 20 positive compounds was analyzed. At 100 μM, only C33 and C97 exerted some toxicity. However, since C97 at this concentration completely inhibited IL-1β, its effect at lower concentrations was studied.

To ensure that NLRP3 inhibition produced the observed IL-1β reduction, IL-1β release upon treatment of microglia from NLRP3 KO mice with LPS and ATP was measured. Neither LPS nor the combination of LPS and ATP, increased the IL-1β concentration in cell supernatants. This fact indicates that LPS-ATP only activates the NLRP3 inflammasome, and does not have any effect on other IL-1β-producing inflammasomes.

The selected compounds inhibit NLRP3 in a concentration-dependent manner, presenting three of them IC₅₀ in the low micromolar range. The dependency of the inhibitory effect of the selected compounds with their concentration was studied next, to identify the most potent NLRP3 inhibitors. The six agents reduced the IL-1β concentration in the supernatant of activated microglia cultures in a concentration-dependent manner. The pretreatment with 100 μM of C61, C75, C78, and C97 completely prevented IL-1β secretion, while the concentration required to reach the 100% of inhibition is lower than 25 μM in the case of C75, C78, and C97 (Table 1). Furthermore, the nonlinear fit of the data allowed the calculation of the IC₅₀ for the studied molecules. According to these data, C75, C78, and C97 were the most potent NLRP3 inhibitors, with IC₅₀ values around 10 μM (Table 1). Then, the toxicity of different concentrations, ranging from 0.01 to 50 μM, of each of the three compounds on microglia cultures was analyzed, and it was found that at 50 μM, none of the compounds is toxic.

TABLE 1
Inhibitory parameters obtained for the six compounds
selected during the secondary in-vitro screening.

| Compound | % Residual | [Cₓ] to reach 100% | |
|---|---|---|---|
| Number | activity* | of inhibition (μM) | IC₅₀ (μM) |
| C56 | 1.4 | >100 | 40.0 ± 2.4 |
| C61 | 0 | ≤50 | 15.4 ± 1.18 |
| C75 | 0 | ≈25 | 9.24 ± 1.06 |
| C78 | 0 | ≤25 | 8.26 ± 1.16 |
| C87 | 14.3 | >100 | 57.8 ± 50 |
| C97 | 0 | ≈25 | 11.27 ± 1.09 |

*Percentage of IL-1β, referred to control, released by microglia cultures pretreated with 100 μM of the corresponding compound before NLRP3 activation with LPS and ATP.

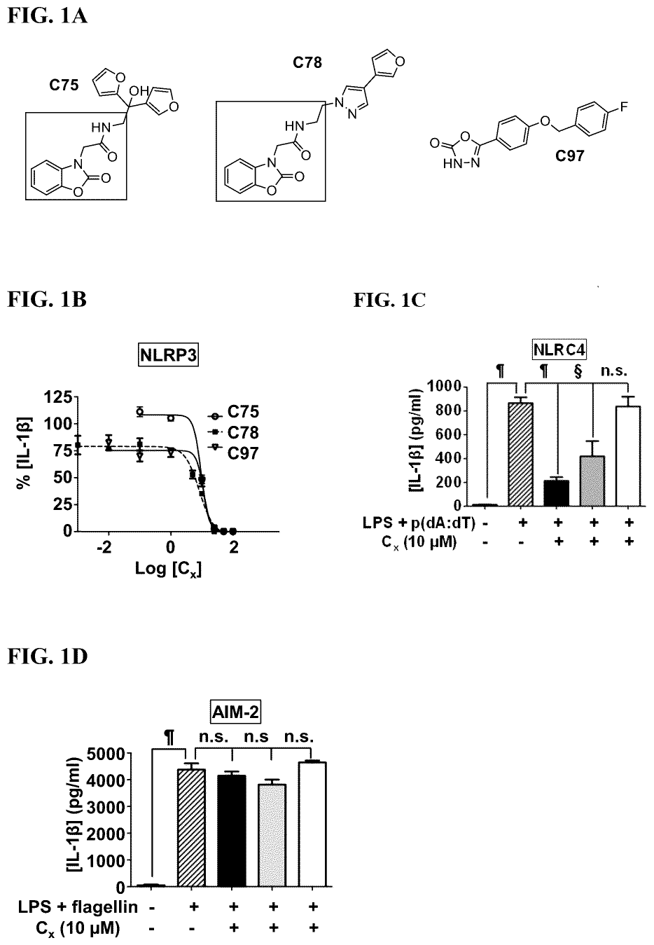

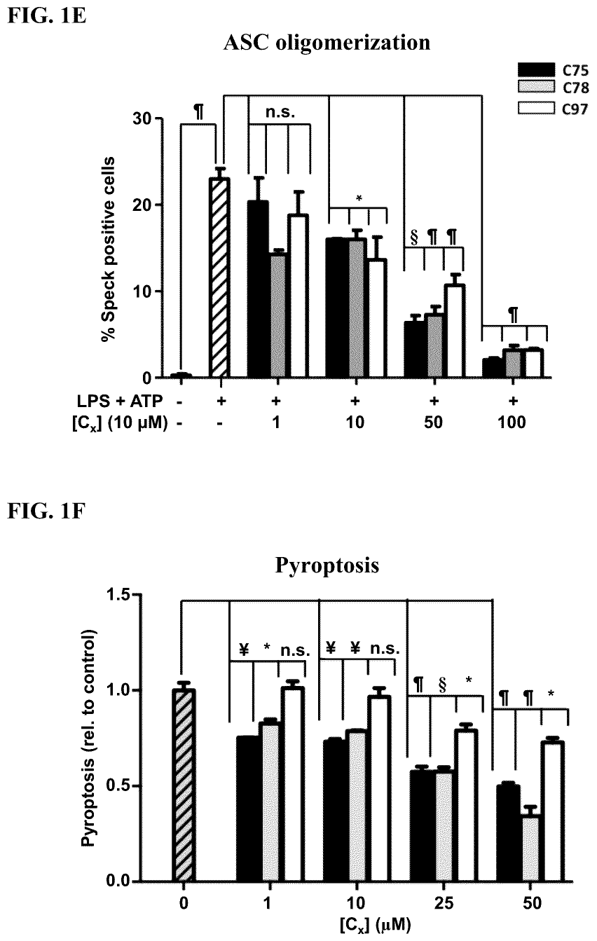

C97 is specific for NLRP3, while C75 and C78 are able to inhibit the NLRC4 activity. FIG. 1. The structure of the three most potent compounds from the in-vitro screening, C75, C78, and C97 are shown in FIG. 1A. The benzoxazolone acetamidyl group shared by C75 and C78 is squared. Dose-response curves for the three compounds are illustrated in FIG. 1B. Nonlinear regression of the data allowed the calculation of IC₅₀ values.

To determine whether the three selected compounds were specific for NLRP3, or they can also inhibit other IL-1β producing inflammasomes, the effect of each molecule on the IL-1β released upon activation of the AIM2 and the NLRC4 inflammasomes was studied. Thus, to increase the expression of procaspase-1, ASC, and pro-IL-1β, murine microglia cultures were primed with LPS, and then, specific activators—double stranded DNA, and flagellin for AIM2 and NLRC4, respectively—were used to activate the corresponding inflammasomes. NLRC4 was primed and specifically activated in microglia from NLRP3 KO mice, using LPS and flagellin, respectively, as shown in FIG. 1C. FIG. 1D illustrates the effect of C75, C78, and C97 on the activity of the AIM2 inflammasome. Microglia cultures from NLRP3 KO mice were primed with LPS, and AIM2 activation was induced by transfection of poly(dA:dT). As shown in FIG. 1C and 1D, C97 was specific for NLRP3, while C75 and C78—at a 10 μM concentration—also presented inhibitory power against the NLRC4 inflammasome. As expected, none of the three compounds inhibited the activity of the AIM2 inflammasome.

C75, C78 and C97 inhibit processes downstream of NLRP3 activation. Next, C75, C78, and C97 were evaluated to determine if they could block processes downstream of NLRP3 activation, such as ASC oligomerization or pyroptosis. ASC oligomerization is necessary for the assembly of the NLRP3 inflammasome, propagates inflammation, and has prionoid properties. Therefore, monitoring ASC oligomerization can be used as an endpoint to determine NLRP3 inflammasome activation. To evaluate the ability of different concentrations of each compound to reduce the percentage of ASC-speck positive cells, murine macrophages that express the ASC protein with a fluorescent tag were used. Murine macrophages expressing m-cerulean tagged ASC were primed and activated with LPS and ATP, respectively. Pretreatment with 10 μM of C75, C78 or C97, significantly reduced the rate of speck positive cells, when compared with DMSO-treated cells. Furthermore, the inhibition of the ASC speck formation proportionally depended on the compound concentration (FIG. 1E). We also examined whether the three compounds inhibit pyroptosis, which is an inflammatory type of programmed cell death. Pyroptosis is located downstream of NLRP3 activation and requires the action of caspase-1. Pyroptosis in primary microglia cultures pretreated with DMSO or different concentrations of the compounds were activated with LPS and ATP. In all panels, C75, C78, and C97 are displayed as black, grey, and white bars, respectively. Reference controls are showed in crosswise lines. All the experiments were carried out by triplicate, while experiments shown in panel E were performed by quadruplicate. Statistical significances are referred to control samples and consist of cells treated with the same DMSO % and same LPS and ATP concentrations as the compound-treated samples. Significance levels are indicated as * p≤0.05, ¥ p≤0.01, § p≤0.001,

¶ p≤0.0001. C75 and C78 dose-dependently inhibited pyroptosis at concentrations starting from 1 μM, while higher C97 concentrations were needed to observe a similar effect (FIG. 1F).

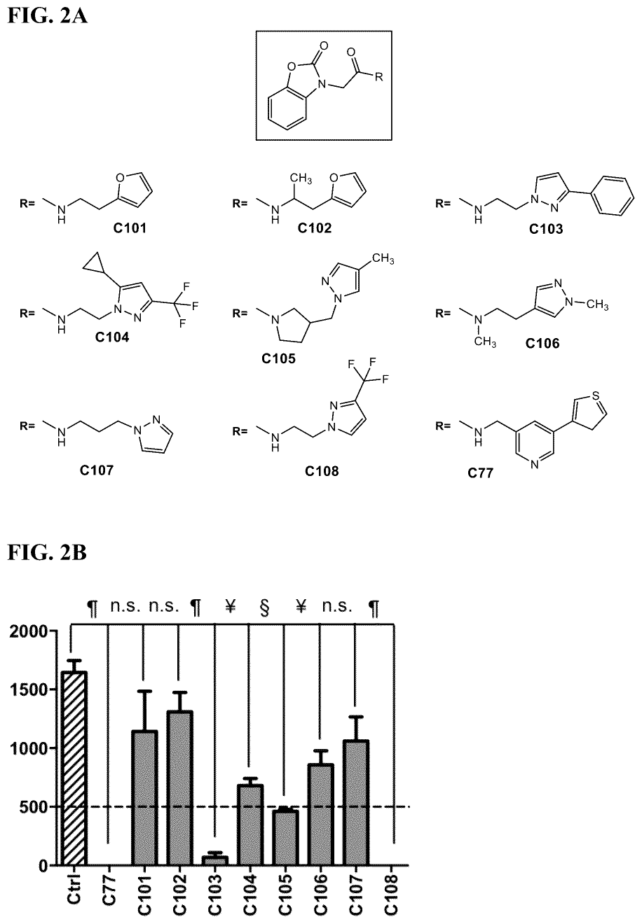

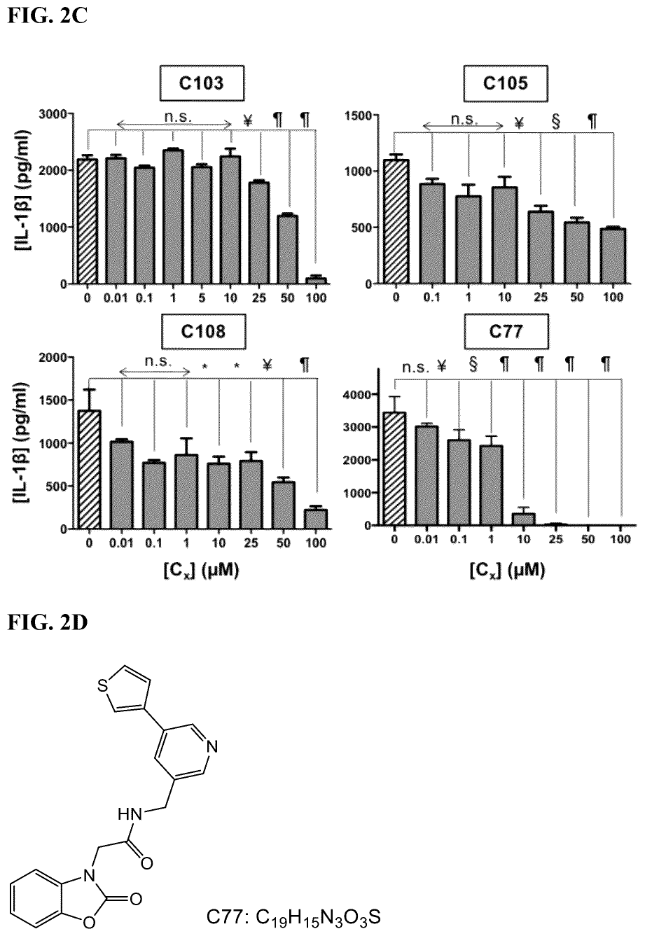

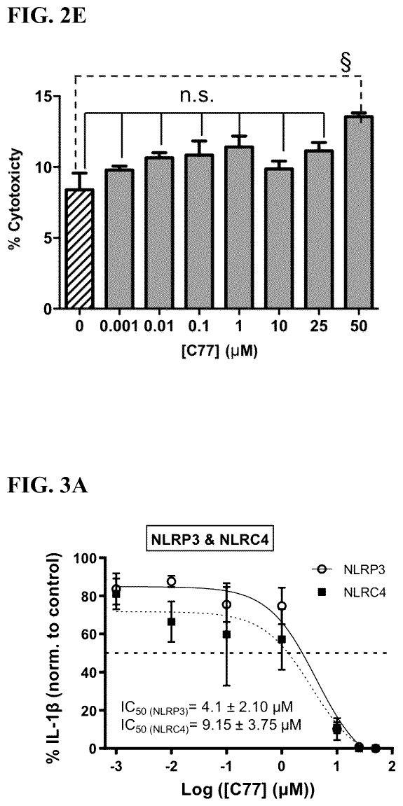

The identification of the active pharmacophore allowed the selection of chemical analogs with NLRP3-inhibitory activity. C75 and C78 present the same chemical core—a benzoxazolone acetamide group (FIG. 1A)—which suggests that this chemical structure is exceptionally efficient for the allocation of the inhibitors within the ATP binding pocket of the NLRP3 protein. Once the potential pharmacophore was identified, a homology search was performed to find chemical analogs of C75 and C78. Thus, using the Molport web server, commercially available compounds that presented the same benzoxazolone acetamide core, and different R substituents (FIG. 2A), were found. FIG. 2A shows the structure of the common core identified as the pharmacophore (in square) and chemical structures of the “R” substituents of the selected benzoxazolone acetamide analogs. Additionally, together with the new compounds, compound C77, included in the 100 screening compounds was tested (but due to delayed delivery, it was not assayed at the same time that the initial hits).

Next, the ability of the analogs to inhibit IL-1β release, in primary microglia cultures, upon NLRP3 activation with LPS and ATP, was assayed. The inhibitory effect of 100 μM of each analog was tested. Six of the analogs—C77, C103, C104, C105, C106, and C108—inhibited NLRP3 with statistical significance (FIG. 2B), which validated the nature of the benzoxazolone acetamide core as the active pharmacophore. Those four compounds that inhibited the IL-1β production below a 30% arbitrary threshold were selected, and dose-response curves were performed to determine their potency (FIG. 2C). Inhibition of NLRP3 activation by different concentrations of the selected analogs, was determined by measuring the IL-10 produced by microglia cultures. Through nonlinear regression, IC₅₀ values of 87.2±2.0, 45.4±9.1, 35.7±5, and 4.10±2.1 μM were obtained for C103, C105, C108, and C77, respectively. Among the evaluated compounds, C77 completely inhibited NLRP3-mediated IL-1β production at concentrations lower than 25 μM, and it also presented the lowest IC₅₀ value among all the evaluated compounds.

C77 is the most potent inhibitor of NLRP3 and NLRC4 inflammasomes, but it does not affect AIM2, IL6 and TNF-α production, or the transcriptional priming of the inflammasome. As aforementioned, C77 was the most promising compound; thus, it was characterized further. C77 contains the benzoxazolone acetamide pharmacophore attached to a tiophenyl-pyridinyl substituent through a methyl linker (FIG. 2D), which leaves the acetamidyl group as a secondary amide. The toxicity of C77 was first evaluated using murine microglia cultures. The compound did not show significant toxicity levels at concentrations lower than 50 μM, although at this concentration, C77 significantly reduced the viability of the culture. Nevertheless, C77 exerted inhibitory effects at concentrations starting from 100 nM (FIG. 2C), thus from this point forward, only compound concentrations lower than 25 μM were used. FIG. 2E illustrates the cytotoxicity exerted by different concentrations of C77 in microglial murine cultures. All the experiments were performed by triplicate. Significance levels are indicated as * p≤0.05, ¥ p≤0.01, § p≤0.001, ¶ p≤0.0001.

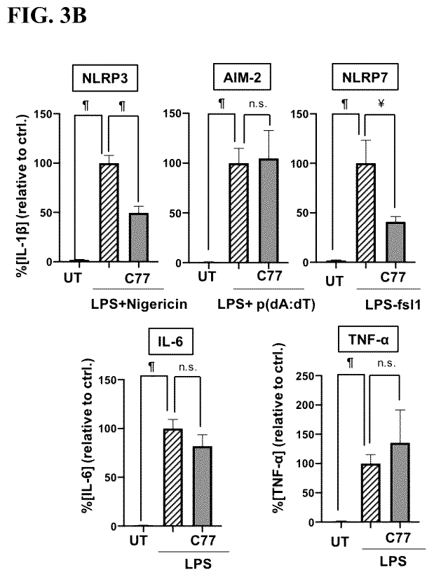

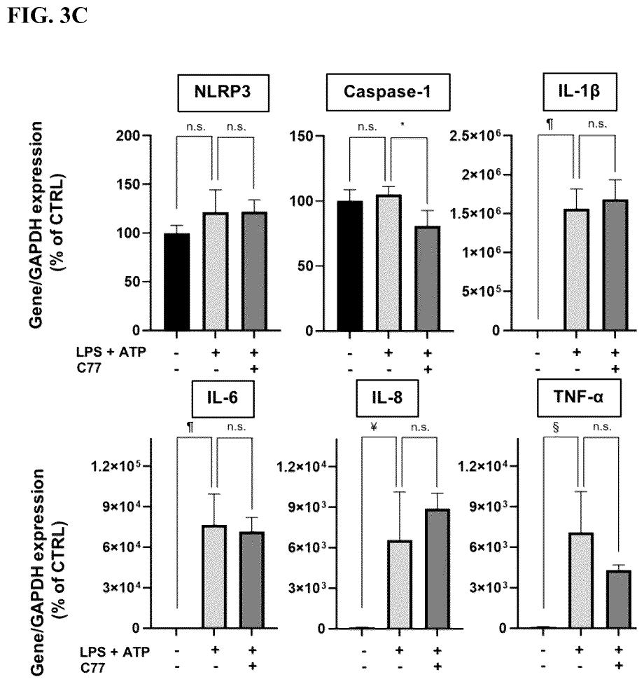

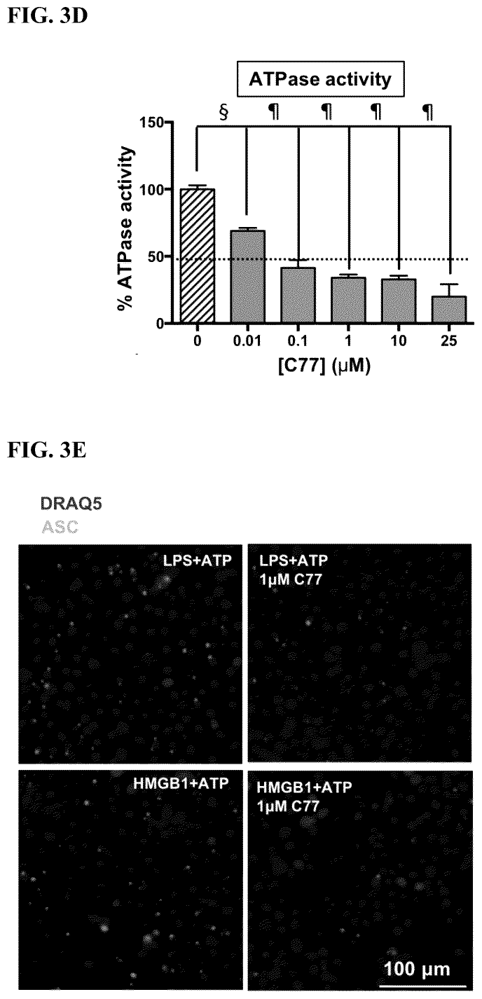

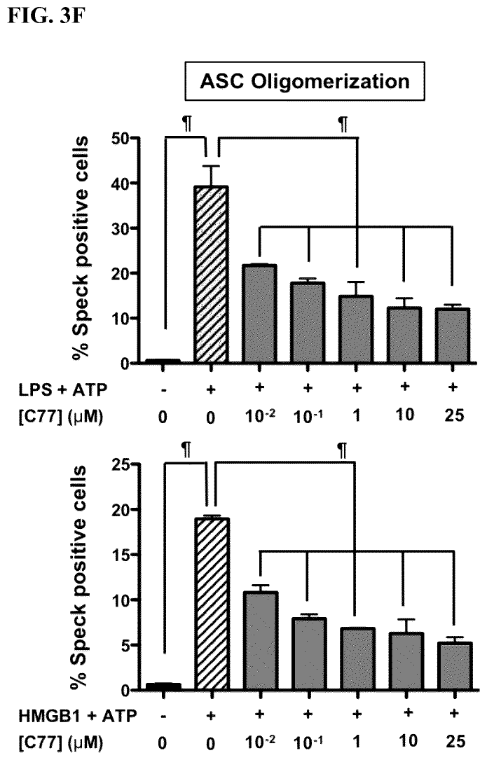

The effect of C77 on the IL-1β producing inflammasomes, NLRC4, NLRP7 and AIM2, and on the production of IL6 and TNF-α, was studied. As shown in FIG. 3A, C77 potently inhibited NLRC4, showing an IC₅₀ value similar to that presented for NLRP3 (NLRP3 IC₅₀=3.54±1.30 μM; NLRC4 IC₅₀=4.15±1.65 μM). It is worthy to notice that C77 did not inhibit IL6, TNF-α production or AIM2-mediated IL-1β release (FIG. 3B). This fact highlights that, as expected, C77 carries out its inhibitory activity through its competent binding to the ATP pocket of NLRP3, NLRC4, and NLRP7 since AIM2 does not present this type of nucleotide-binding site. In addition, C77 did not modify the mRNA levels of components of inflammatory cytokines, such as IL-6, IL-8, or TNF-α, and did not affect the transcriptional priming of NLRP3 or IL-1β, although it slightly reduced the expression of caspase-1 (FIG. 3C).

C77 dose-dependently inhibits NLRP3 ATPase activity and downstream processes. The ability of C77 to reduce the NLRP3 activity at the protein level was analyzed. Thus, the isolated protein was incubated with different concentrations of the compound before adding the ATP substrate. C77 dose-dependently inhibited the NLRP3 ATPase activity, showing statistically significant inhibition at concentrations as low as 10 nM (FIG. 3D). The nonlinear fit of the data allowed the estimation of an IC₅₀ value for the ATPase activity of 40 nM.

Once it was demonstrated that the compound directly inhibited the NLRP3 catalytic activity, the impact of C77 on ASC speck formation was analyzed. In this case, the response of ASC m-cerulean murine macrophages in response to two different activation stimuli was studied; thus, NLRP3 activation both with LPS and ATP was induced, as well as with HMGB1 and ATP (FIG. 3E). HMGB1 is an alarmin released under stress situations, and it acts as a danger-associated molecular pattern (DAMP), activating different inflammatory pathways, the NLRP3 inflammasome among them. Therefore, with this experiment, the competency of C77 to inhibit NLRP3 activation in response to both PAMPs and DAMPs was demonstrated. The reduction of the speck positive cells depended on the compound concentration in a dose-dependent manner, being the inhibitory effect observable at concentrations starting from 10 nM (FIG. 3F).

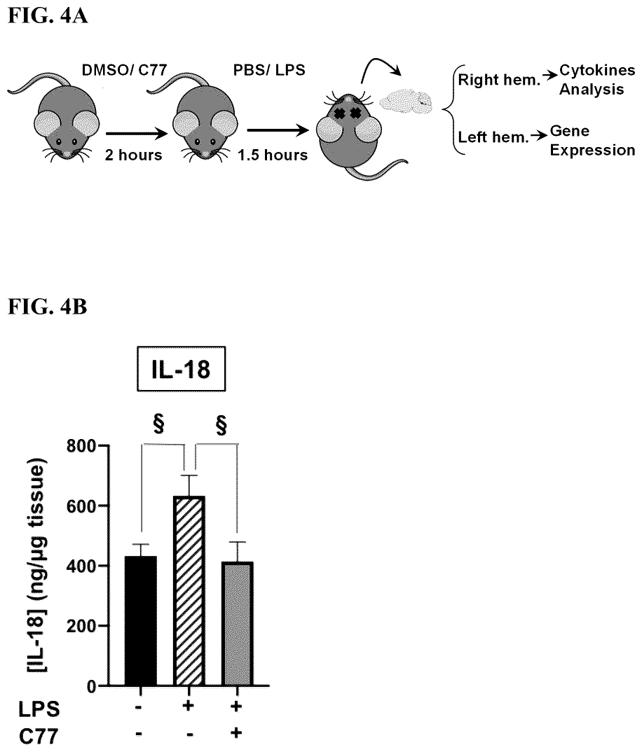

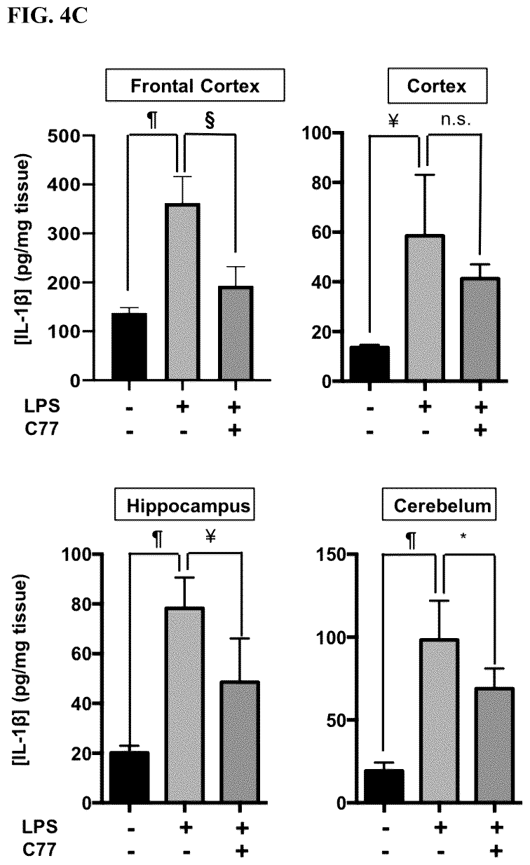

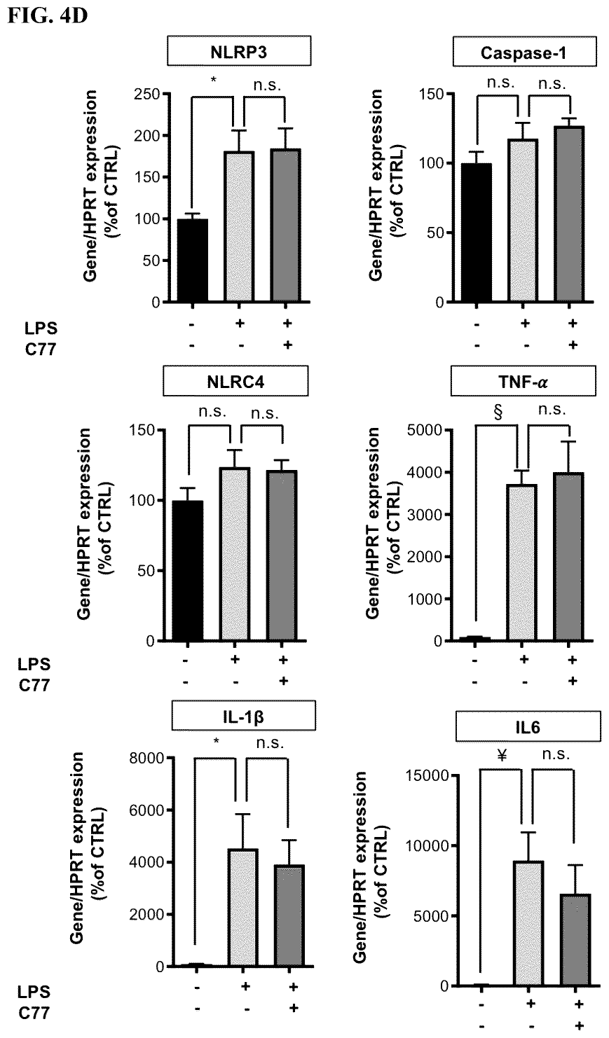

C77 is effective in-vivo, reducing IL-1,8 in brain sections of LPS-treated mice, but it does not act on inflammasome priming. The performance of C77 in-vivo was evaluated. For that purpose, mice were intraperitoneally (IP) administered 50 mg/kg of C77 or vehicle (DMSO), 2 hours before IP administration of 15 mg/kg of LPS or vehicle (PBS). Different brain sections were dissected, and the IL-1β quantity was analyzed (FIG. 4A). As shown in FIG. 4B, IP administrations of LPS significantly increased the IL-18 amount in the frontal cortex while pretreatment with C77 significantly reduced IL-18 levels. As shown in FIG. 4C, IP administration of LPS significantly increased the IL-1β amount in the brain, while pretreatment with C77 significantly reduced IL-1β levels in the frontal cortex, hippocampus, and cerebellum of LPS-treated mice. Additionally, it was confirmed by western blot that C77 reduces the cleaved form of IL-β and not pro IL-1β. It was also evaluated whether C77 might act on the inflammasome priming step. Thus, the RNA expression levels of inflammasome-related genes, such as NLRP3, caspase-1, IL-1β, and TNF-α, were studied. Additionally, the C77 effect on IL6 and NLRC4 expression was examined. LPS treatment significantly increased NLRP3, IL1β, TNF-α and IL6 expression in mouse brains. FIG. 4D shows the RNA expression levels of inflammatory genes in the brain of mice treated with vehicle (DMSO), or C77 before PBS, or LPS IP injection. n=5 animals per group. Pretreatment with 50 mg/kg of the compound 2 hours before LPS administration did not reduce the overexpression of these genes (FIG. 4D), indicating that, as expected, C77 in-vivo inhibited the inflammasome activation and not the priming stage.

Compounds C75, C78, and C97, which inhibit NLRP3-activation, as well as downstream processes were identified. C75 and C78 share the same chemical core, which suggests that this structure might be particularly useful for the allocation of the compounds within the ATP-binding site, revealing the benzoxazolone acetamidyl group as the potential pharmacophore. To explore this hypothesis, chemical analogs that contain this group were identified. Around 67% of the assayed compounds significantly reduced the activity of the NLRP3 inflammasome, which validates the nature of the proposed pharmacophore. To elucidate the structural basis behind the differences in the performance of the analogs, computational dockings were carried out to identify critical elements for NLRP3-drug interactions. The dockings revealed that the benzoxazolone acetamidyl core interacts with the same residues that stabilize the ATP adenine ring (I18 and I234), suggesting that this group anchors the molecule in the right position within the binding site. Interestingly, although all analogs keep these interactions, only those that also interact with K232 and T233 present in-vitro activity, being exceptionally efficient the analogs that have an aromatic heterocycle with an electronegative heteroatom at an adequate distance from the pharmacophore—C75, C77, and C78. Therefore, the positively charged K232 might interact with the partial negative charge of the heteroatom, or with the partial negative charges of the carbons—stablished through resonant structures—stabilizing the compound in the ATP pocket. Consequently, the length of the linker is capital to allow the interaction of the heterocycle with K232, being apparently the optimal distance between the core and the electronegative heteroatom of 9 bonds. These findings are relevant not only for this current project but also for future works that aim to deepen in the action mechanism of the NLRP3 protein at the molecular level.

Among the chemical analogs, C77 appeared as the most promising agent. This compound inhibits NLRP3 and NLRC4 with similar potency, but it does not affect AIM2 or IL6 pathways. AIM2 does not have a NACHT domain, and neither this motive is involved in IL6 synthesis; therefore, the data suggest that C77 inhibits both inflammasomes through its direct binding to the ATP pocket. This hypothesis was further demonstrated by showing—at the protein level—the concentration-dependent inhibition of the NLRP3 ATPase activity. C77 also inhibits ASC oligomerization in response to both sterile and pathogen stimuli. Considering the impact that ASC specks have on different neurological disorders, and especially in AD, C77 might have therapeutic effects in this condition. Finally, C77 also reduces LPS-induced inflammation in different brain parts, which suggests that the agent might penetrate the brain-blood barrier, being potentially useful for reducing the neuroinflammation inherent to neurological disorders.

Three compounds exhibited submicromolar IC₅₀ values, possess a tolerable preliminary toxicity profile, and inhibit formation of ASC specks in response to NLRP3-specific agonists. The synthesis of analogs of these compounds and for the development of structure-activity relationships of C77 will be undertaken. Preliminary structure-activity relationships (SAR) by chemical similarity searches based on the C77 benzoxazolone core were undertaken, and studied and the four new analogs below had activity as demonstrated using the in vitro assay:

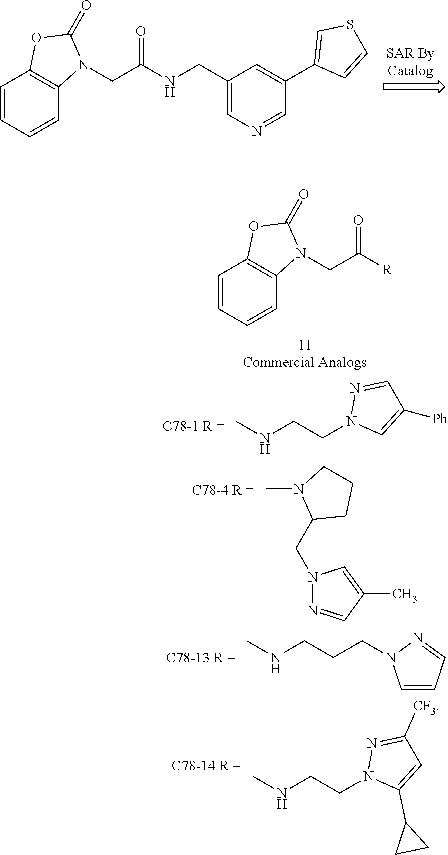

Preparation of compounds can involve the protection and deprotection of various chemical groups. The need for protection and deprotection, and the selection of appropriate protecting groups, can be readily determined by one skilled in the art. Suitable groups for that purpose are discussed in standard textbooks in the field of chemistry, such as Protective Groups in Organic Synthesis by T. W. Greene and P. G. M. Wuts [John Wiley & Sons, New York, 1999], in Protecting Group Chemistry, 1^{st} Ed., Oxford University Press, 2000; and in March's Advanced Organic chemistry: Reactions, Mechanisms, and Structure, 5^{th} Ed., Wiley-Interscience Publication, 2001.

Many compounds described herein may be prepared by the schemes below. Compound C77 and related analogs can be synthesized as shown in the schemes below.

Benzoxazolone Amine Library Synthesis SAR Plans: Synthesis A shows the synthetic chemistry that can be utilized to efficiently expand the carboxamide SAR using a rapid analog synthesis library chemistry approach. Known benzoxazolone intermediate can be made in one pot by simple benzoxazolone N-alkyation with bromoacetic acid ester followed by saponification. The carboxylic acid can be coupled using standard amide formation procedures with a wide variety of commercially available amines to efficiently produce SAR, where amine substituents R¹ and R² are modified.

### Synthesis A:

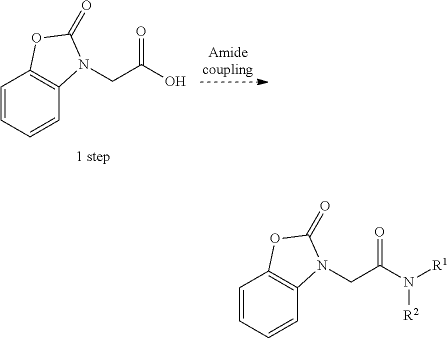

Single specific changes to benzoxazolone scaffold can be pursued as outlined in Synthesis B.

### Synthesis B:

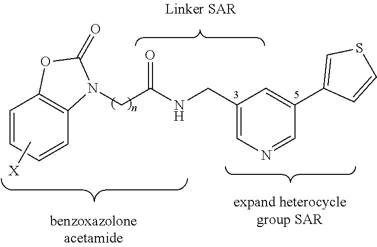

General Synthetic Route for additional Benzoxazolone Analogs: Compounds can be synthesized using standard organic chemistry techniques as shown in Synthesis C.

### Synthesis C:

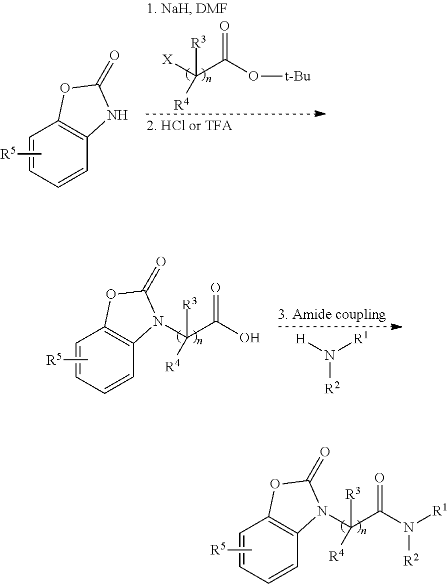

Benzoxazolone starting materials with a wide variety of substituents X shown at the left of the scheme can be purchased commercially or made in one step for 1,2-aminophenols using carbonyl diimidazole (not shown). Standard N-alkylation proceeds using sodium hydride base followed by addition of readily available haloalkyl carboxylic ester reagents, using, for example, t-butyl esters. The subsequent products are then treated with acid to produce the carboxylic acid intermediate shown in the center of the scheme. Standard carboxylic acid-amine coupling techniques can be used to produce benzoxazolone carboxamide final compounds with various substituents R⁵, linker modifications R³, R⁴, n and amine substituents R¹ and R².

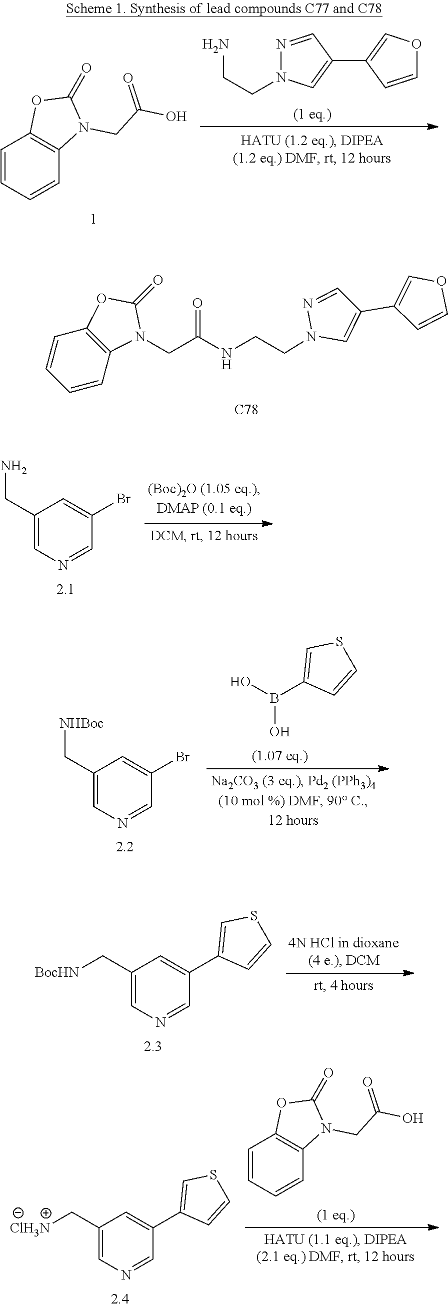

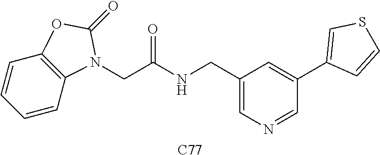 [SMILES: O=C(Cn1c(=O)oc2ccccc21)NCc1cncc(-c2ccsc2)c1 ; "C77"]

### Re-synthesis of Lead Compounds C78 and C77:

Lead compounds C78 and C77 were synthesized by the protocol outlined in Scheme 1. (2-oxo-1,3-benzoxazol-3(2H)-yl)acetic acid 1 underwent amide coupling with 2-(4-(furan-3-yl)-1H-pyrazol-1-yl)ethan-1-amine in presence of HATU as coupling reagent to afford the desired compound C78 in 71% percent yield as white solid. Re-synthesis of C77 was completed in four steps. First, (5-Bromopyridin-3-yl)methanamine 2.1 was Boc protected followed by the Suzuki coupling with thiophen-3-ylboronic acid to give compound 2.3 as white solid in 89% yield. Acid catalyzed Boc deprotection of 2.3 followed by amide coupling with (2-oxo-1,3-benzoxazol-3(2H)-yl)acetic acid 1 generated the desired compound C77 as white solid in 50% yield.

### Experimental Procedure

Materials and Methods. ¹H were acquired on a Bruker DRX-400 spectrometer at 400 MHz for ¹H. TLC was performed on silica coated aluminum sheets (thickness 200 μm) or alumina coated (thickness 200 μm) aluminum sheets supplied by Sorbent Technologies and column chromatography was carried out on Teledyne ISCO combiflash equipped with a variable wavelength detector and a fraction collector using a RediSep Rf high performance silica flash columns by Teledyne ISCO. LCMS/HPLC analysis for purity and HRMS was conducted on an Agilent Technologies G1969A high-resolution API-TOF mass spectrometer attached to an Agilent Technologies 1200 HPLC system. Samples were ionized by electrospray ionization (ESI) in positive mode. Chromatography was performed on a 2.1×150 mm Zorbax 300SB-C18 5-μm column with water containing 0.1% formic acid as solvent A and acetonitrile containing 0.1% formic acid as solvent B at a flow rate of 0.4 mL/min. The gradient program was as follows: 1% B (0-1 min), 1-99% B (1-4 min), and 99% B (4-8 min). The temperature of the column was held at 50 ° C. for the entire analysis. The chemicals and reagents were purchased from Acros organics, Alfa Aesar, Enamine and Combi-Blocks. All solvents were purchased in anhydrous from Acros Organics and used without further purification.

N-(2-(4-(furan-3-yl)-1H-pyrazol-1-yl)ethyl)-2-(2-oxobenzoidloxazol-3(2H)-yl)acetamide (C77):

To a solution of (2-oxo-1,3-benzoxazol-3(2H)-yl)acetic acid 1 (Enamine) (20 mg, 0.10 mmol), 2-(4-(furan-3-yl)-1H-pyrazol-1-yl)ethan-1-amine (Enamine) (18.3 mg, 0.10 mmol) and HATU (47.2 mg, 0.12 mmol) in DMF (4 mL) was added DIPEA (0.02 mL, 0.12 mmol) and stirred at room temperature for 12 hours. After completion of the reaction monitored by TLC, the reaction mixture was diluted with ethyl acetate (50 mL) and transferred to separatory funnel and washed with water three times (50 mL). The organic layer was collected, dried over magnesium sulfate, filtered, evaporated to get crude mixture which was purified by flash column chromatography using 5% MeOH/DCM as eluent to get the desired product C78 as white solid (26 mg, 71%). ¹H-NMR (400 MHz, d6-DMSO): δ 8.46 (t, J=5.6 Hz, 1H), 7.91 (s, 1H), 7.88 (s, 1H), 7.69 (s, 1H), 7.67 (t, J=1.6 Hz, 1H), 7.34 (m, 1H), 7.10 (m, 3H), 6.73 (m, 1H), 4.45 (s, 2H), 4.18 (t, J=6 Hz, 2H), 3.49 (m, J=5.6 Hz, 2H); HRMS (ESI): m/z [M+H]+ calculated for C18H17N4O4⁺: 353.1244, found: 353.1247; Purity>97%

(5-Bromo-pyridin-3-ylmethyl)-carbamic Acid Tert-butyl Ester (2.2):

To a solution of (5-Bromopyridin-3-yl)methanamine 2.1 (Combi-Blocks) (635 mg, 3.39 mmol) and di-tent-butyl dicarbonate (778 mg, 3.56 mmol) in dichloromethane (10 mL) was added catalytic amount of DMAP (42 mg, 0.33 mmol) and stirred at room temperature overnight. After completion of the reaction monitored by TLC, solvent was evaporated and the crude mixture was purified by column chromatography using 50% ethyl acetate/hexanes as eluent to get the desired product 2.2 as white solid (702 mg, 72%). ¹H-NMR (400 MHz, d6-DMSO): δ 8.58 (d, J=2 Hz, 1H), 8.44 (d, J=1.6 Hz, 1H), 7.87 (m, 1H), 7.48 (t, J=5.6 Hz, 1H), 4.15 (d, J=5.6 Hz, 2H), 7.34 (m, 1H), 7.10 (m, 3H), 6.73 (m, 1H), 4.45 (s, 2H), 4.18 (t, J=6 Hz, 2H), 3.49 (m, J=5.6 Hz, 2H), 1.38 (s, 9H); MS (ESI) m/z 287.03 (M+H)+.

(5-(3-thiophen-1-yl)-pyridin-3-ylmethyl)-carbamic Acid Tert-butyl Ester (2.3):

A solution of (5-Bromo-pyridin-3-ylmethyl)-carbamic acid tert-butyl ester 2.2 (236 mg, 0.88 mmol), thiophen-3-ylboronic acid (Alfa Aesar) (121 mg, 0.94 mmol) and tetrakis(triphenylphosphine)palladium(0) (95 mg, 0.08 mmol) in DMF (3 mL) was stirred at room temperature for 10 minutes. To the above reaction mixture was added 1 molar aqueous solution of sodium carbonate (2.47 mL, 2.47 mmol) and heated to 90° C. overnight. After completion of the reaction monitored by TLC, the reaction mixture was diluted with ethyl acetate (50 mL) and transferred to separatory funnel and washed with water three times (50 mL). The organic layer was collected, dried over magnesium sulfate, filtered, evaporated to get crude mixture which was purified by flash column chromatography using 50% ethyl acetate/hexanes as eluent to get the desired product 2.3 as white solid (213 mg, 89%). ¹H-NMR (400 MHz, CDCl3): δ 8.77 (s, 1H), 8.45 (s, 1H), 7.80 (m, 1H), 7.53 (m, 1H), 7.45 (m, 1H), 7.38 (m, 1H), 4.92 (bs, 1H), 4.39 (d, J=5.6 Hz, 2H), 1.47 (s, 9H); MS (ESI) m/z 291.11 (M+H)+.

(5-(thiophen-3-yl)pyridin-3-yl)methanamine Hydrochloride (2.4):

To a solution of (5-(3-thiophen-1-yl)-pyridin-3-ylmethyl)-carbamic acid tert-butyl ester 2.3 (213 mg, 0.73 mmol) in dichloromethane (3 mL) was added 4 N hydrochloric acid solution in 1,4-dioxane (0.734 mL, 2.93 mmol) and stirred at room temperature for 4 hours. After completion of the reaction monitored by LCMS, solvent was evaporated to give the desired product 2.4 as white solid (200 mg, 88%). ¹H-NMR (400 MHz, d6-DMSO): δ 9.17 (d, J=2.4 Hz, 1H), 8.84 (bs, 1H), 8.77 (d, J=2 Hz, 1H), 8.72 (bs, 2H), 8.22 (m, 1H), 7.80 (m, 1H), 7.76 (m, 1H), 4.22 (m, J=5.6 Hz, 2H); MS (ESI) m/z 191.06 (M+H)+.

2-(2-oxobenzo[d]oxazol-3(2H)-yl)-N-45-(thiophen-3-yl)pyridin-3-yl)methyl)acetamide (C77):

To a solution of (2-oxo-1,3-benzoxazol-3(2H)-yl)acetic acid (Enamine) 1 (26 mg, 0.13 mmol), (5-(thiophen-3-yl)pyridin-3-yl)methanamine hydrochloride 2.4 (30 mg, 0.13 mmol) and HATU (56 mg, 0.15 mmol) in DMF (2 mL) was added DIPEA (0.05 mL, 0.28 mmol) and stirred at room temperature for 12 hours. After completion of the reaction monitored by TLC, the reaction mixture was diluted with ethyl acetate (50 mL) and transferred to separatory funnel and washed with water three times (50 mL). The organic layer was collected, dried over magnesium sulfate, filtered, evaporated to get crude mixture which was purified by flash column chromatography using 5% MeOH/DCM as eluent to get the desired product C77 as white solid (26 mg, 53%). ¹H-NMR (400 MHz, d6-DMSO): δ 8.89 (t, J=5.6 Hz, 1H), 8.86 (d, J=2 Hz, 1H), 8.40 (d, J=2 Hz, 1H), 8.01 (m, 1H), 7.95 (t, J=2 Hz, 1H), 7.73 (m, 1H), 7.62 (m, 1H), 7.37 (m, 1H), 7.25 (m, 1H), 7.16 (m, 2H), 4.59 (s, 2H), 4.41 (d, J=5.6 Hz, 2H); HRMS (ESI): m/z [M +H]+ calculated for C19H16N3O3S+: 366.0907, found: 366.0912; Purity>96%

### Compound Purity Analysis Using HPLC Method:

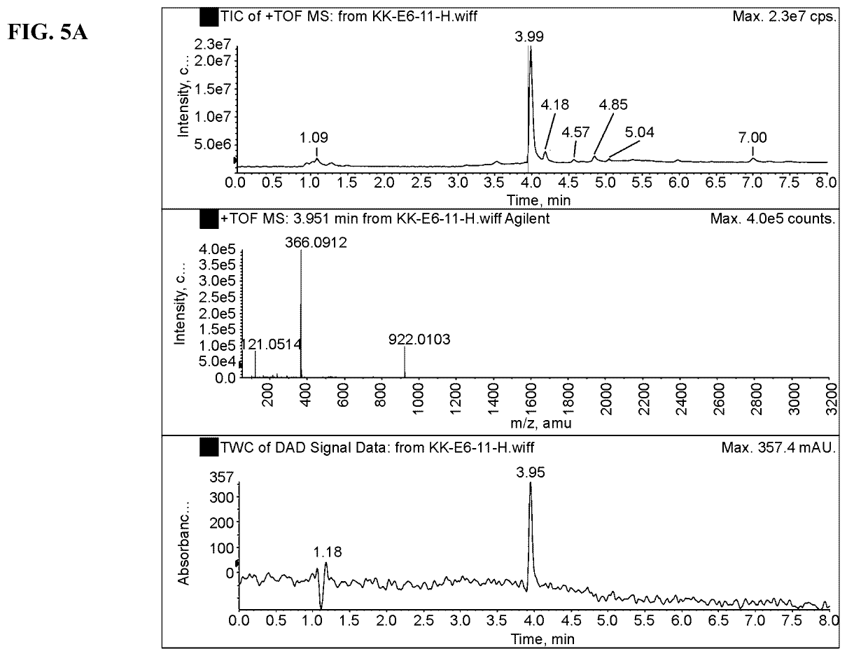

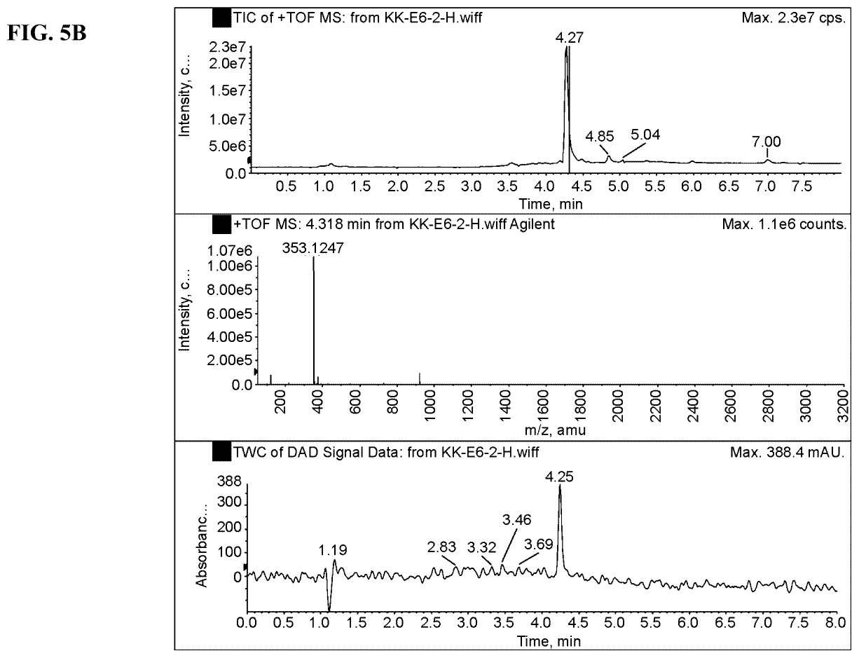

HPLC analysis for purity was conducted on an Agilent Technologies G1969A high resolution API-TOF mass spectrometer attached to an Agilent Technologies 1200 HPLC system. Samples were ionized by electrospray ionization (ESI) in positive mode. Chromatography was performed on a 2.1×150 mm Zorbax 300SB-C18 5-μm column with water containing 0.1% formic acid as solvent A and acetonitrile containing 0.1% formic acid as solvent B at a flow rate of 0.4 mL/min. The gradient program was as follows: 1% B (0-1 min), 1-99% B (1-4 min), and 99% B (4-8 min). The temperature of the column was held at 50° C. for the entire analysis. The HPLC chromatogram for Compounds C77 and C78 are shown in FIG. 5A and FIG. 5B, respectively.

### EMBODIMENTS

Various non-limiting embodiments [A] to [T] of the invention can be described in the text below:

[Embodiment A] A compound of formula I:

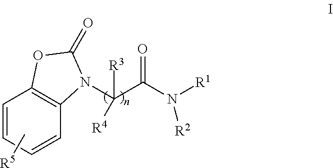

wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
wherein the following compounds are excluded:

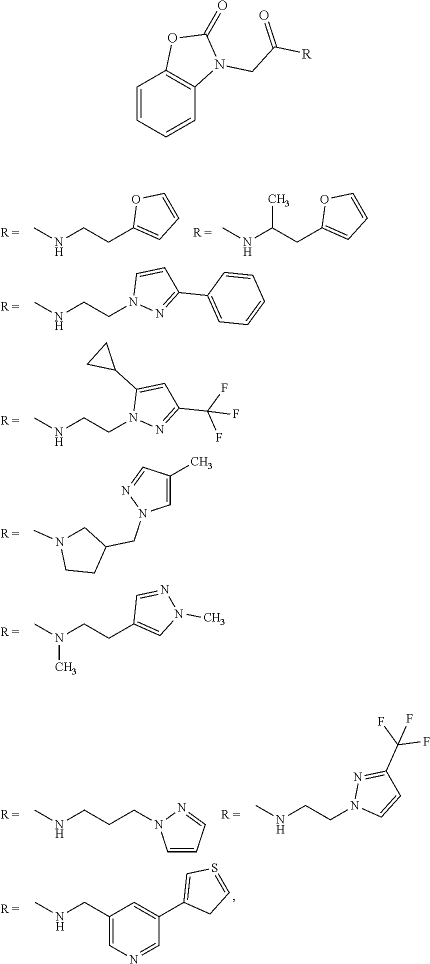

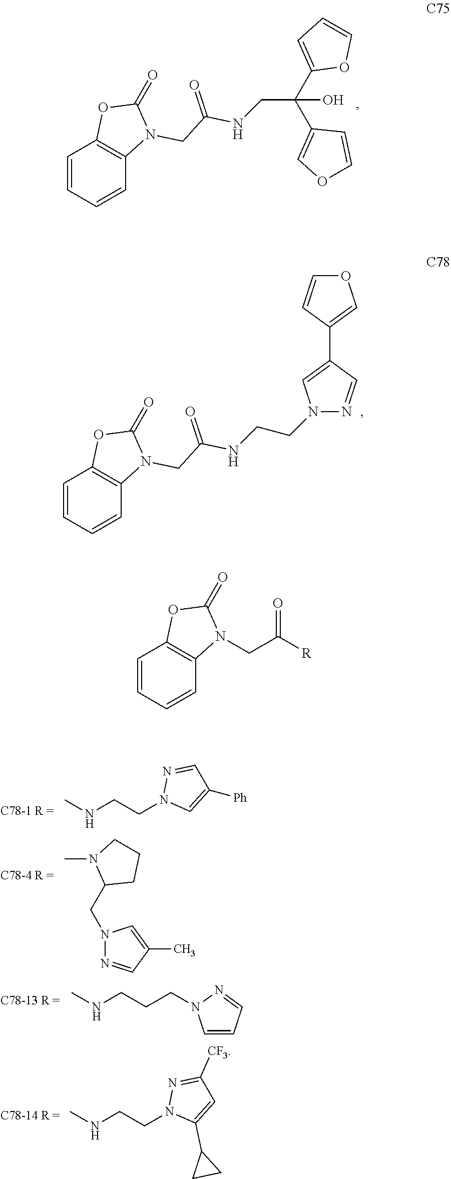

[Embodiment B] A compound according to Embodiment [A] above, or according to other embodiments of the invention, wherein n is 1.

[Embodiment C] A compound according to Embodiment [A] or [B] above, or according to other embodiments of the invention, wherein R³ and R⁴ are independently selected in each instance from hydrogen and methyl.

[Embodiment D] A compound according to any one of Embodiments [A]-[C] above, or according to other embodiments of the invention, wherein R³ and R⁴ are each hydrogen.

[Embodiment E] A compound according to any one of Embodiments [A]-[D] above, or according to other embodiments of the invention, wherein R⁵ is hydrogen in each instance.

[Embodiment F] A compound according to any one of Embodiments [A]-[E] above, or according to other embodiments of the invention, wherein R² is hydrogen or methyl.

[Embodiment G] A compound according to any one of Embodiments [A]-[F] above, or according to other embodiments of the invention, wherein R² is hydrogen.

[Embodiment H] A compound according to any one of Embodiments [A]-[E] above, or according to other embodiments of the invention, wherein R¹ and R², together with the nitrogen to which they are attached, form an optionally substituted heterocyclyl.

[Embodiment I] A compound according to any one of Embodiments [A]-[E], and H above, or according to other embodiments of the invention, wherein R¹ and R², together with the nitrogen to which they are attached, form an optionally substituted pyrrolidine, piperidine, azetidine, azepine, piperazine, morpholine, pyrroline, imidazoline, imidazolidine, pyrazoline, pyrazolidine.

[Embodiment J] A compound according to any one of Embodiments [A]-[I] above, or according to other embodiments of the invention, wherein p is selected from 1, 2, or 3.

[Embodiment K] A compound according to any one of Embodiments [A]-[J] above, or according to other embodiments of the invention, wherein R^{a} and R^{b} are each hydrogen.

[Embodiment L] A compound according to any one of Embodiments [A]-[K] above, or according to other embodiments of the invention, wherein R¹⁰ is selected from hydrogen, phenyl, methyl, cyclopropyl, trifluoromethyl, thiophene, or furan.

[Embodiment M] A pharmaceutical composition comprising a compound of formula I and a pharmaceutically acceptable excipient or carrier:

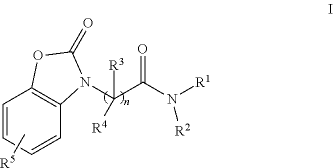

wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;

R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
RC is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

[Embodiment N] A method of treating a neuroinflammatory disorder in a subject, comprising administering to the subject a therapeutically effective amount of a compound of formula I:

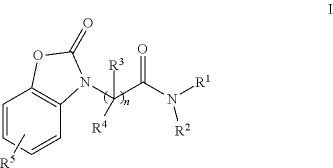

wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

[Embodiment O] A method for treating a neuroinflammatory disorder involving the dysregulation of one or more NLRs, the method comprising administering to a subject a therapeutically effective amount of a compound of formula I:

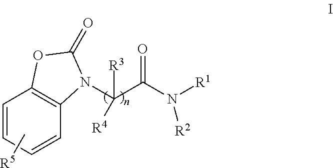

wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO2NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

[Embodiment P] The method according to Embodiment [N] or [O] above, or according to other embodiments of the invention, wherein the neuroinflammatory disorder is selected from an autoimmune disease, type-2 diabetes, a Cryopyrin-Associated Autoniftafnmatory Syndrome (CAPS), Alzheimer's disease (AD), Parkinson's disease (PD), Amyotrophic Lateral Sclerosis (ALS), Multiple Sclerosis (MS), and rheumatoid arthritis (RA).

[Embodiment Q] A method for inhibiting the formation of inflammasomes, comprising exposing a cell to an effective amount of a compound of formula I:

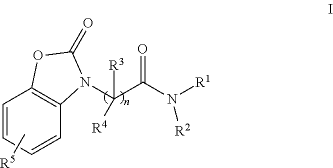

wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C4)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
Rₐ and R_{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)Rₐ, —C(O)OR^{a}, —SR^{a}, —SO₂R^{a}, —SO₂NR^{a}R^{b}, aryl, and heterocyclyl.

[Embodiment R] A method for inhibiting the activation of NLRP3 or NLRC4 receptors, comprising exposing a cell to an effective amount of a compound of formula I:

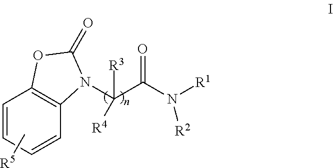

wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

[Embodiment S] An in vitro method according to any one of Embodiments [N]-[R] above, or according to other embodiments of the invention.

[Embodiment T] An in vivo method according to any one of Embodiments [N]-[R] above, or according to other embodiments of the invention.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A compound of formula I: 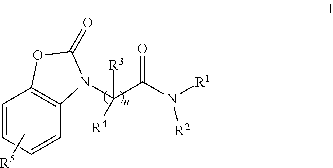
wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR_{c}, —C(O)R^{a}, —OC(O)Rₐ, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
wherein the following compounds are excluded:

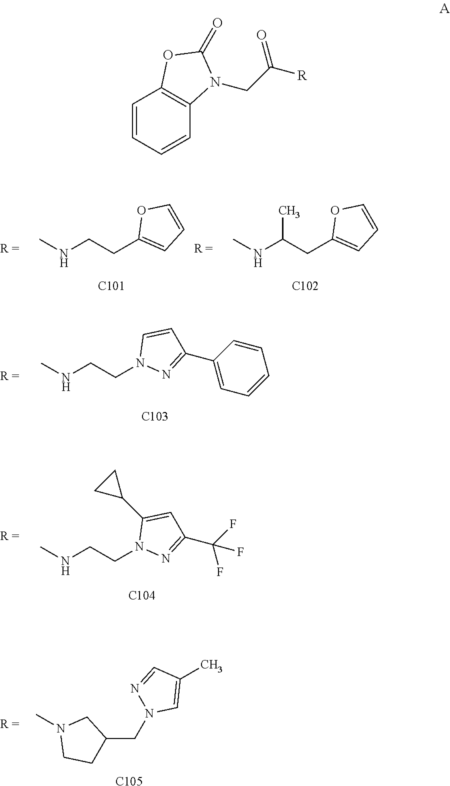

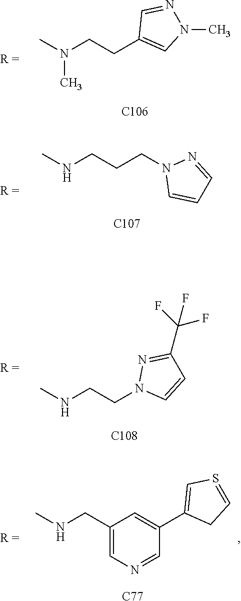

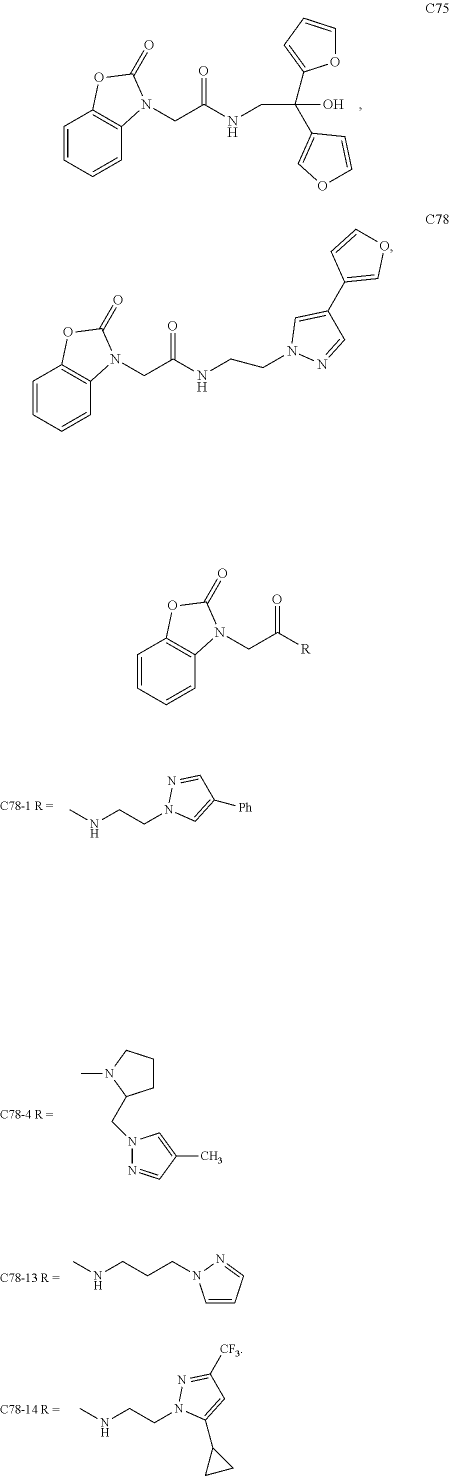

2. A compound according to claim 1, wherein n is 1.

3. A compound according to claim 1, wherein R³ and R⁴ are independently selected in each instance from hydrogen and methyl.

4. A compound according to claim 3, wherein R³ and R⁴ are each hydrogen.

5. A compound according to claim 1, wherein R⁵ is hydrogen in each instance.

6. A compound according to claim 1, wherein R² is hydrogen or methyl.

7. A compound according to claim 6, wherein R² is hydrogen.

8. A compound according to claim 1, wherein R¹ and R², together with the nitrogen to which they are attached, form an optionally substituted heterocyclyl.

9. A compound according to claim 8, wherein R¹ and R², together with the nitrogen to which they are attached, form an optionally substituted pyrrolidine, piperidine, azetidine, azepine, piperazine, morpholine, pyrroline, imidazoline, imidazolidine, pyrazoline, pyrazolidine.

10. A compound according to claim 1, wherein p is selected from 1, 2, or 3.

11. A compound according to claim 1, wherein R^{a} and R^{b} are each hydrogen.

12. A compound according to claim 1, wherein R¹⁰ is selected from hydrogen, phenyl, methyl, cyclopropyl, trifluoromethyl, thiophene, or furan.

13. A pharmaceutical composition comprising a compound of formula I and a pharmaceutically acceptable excipient or carrier: 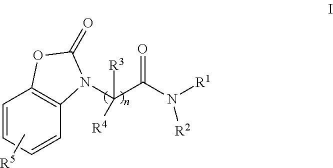
wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

14. A method of treating a neuroinflammatory disorder in a subject, comprising administering to the subject a therapeutically effective amount of a compound of formula I: 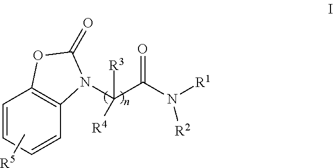
wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C¹-C⁴)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SRₐ, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

15. A method for treating a neuroinflammatory disorder involving the dysregulation of one or more NLRs, the method comprising administering to a subject a therapeutically effective amount of a compound of formula I: 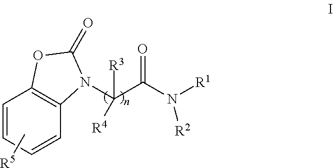
wherein:
R¹ is —(CR^{a}R^{b})ₚ-Q-R¹⁰,
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})^{p}-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

16. The method according to claim 14 or 15, wherein the neuroinflammatory disorder is selected from an autoimmune disease, type-2 diabetes, a Cryopyrin-Associated Autoinflarnmatory Syndrome (CAPS), Alzheimer's disease (AD), Parkinson's disease (PD), Amyotrophic Lateral Sclerosis (ALS), Multiple Sclerosis (MS), and rheumatoid arthritis (RA).

17. A method for inhibiting the formation of inflammasomes, comprising exposing a cell to an effective amount of a compound of formula I: 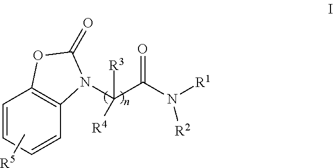
wherein:
R¹ is —(CR^{a}R^{b})ₚ-Q-R¹⁰;
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, —OR^{a}, (C₁-C4)haloalkoxy, (C₁-C₄)alkylthio, —(C₁-C₆)haloalkylthio, halogen, cyano, nitro, (C₁-C₄)alkylsulfonyl, —NR^{a}R_{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C4)alkylthio, —(C₁-C₆)haloalkylthio, halogen, cyano, nitro, (C₁-C4)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

18. A method for inhibiting the activation of NLRP3 or NLRC4 receptors, comprising exposing a cell to an effective amount of a compound of formula I: 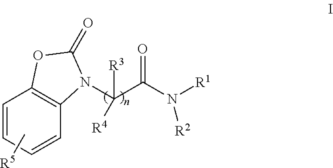
wherein:
R¹ is —(CR³R^{b})ₚ-Q-R¹⁰,
R² is hydrogen or optionally substituted (C₁-C₈)hydrocarbon; or
R¹ and R², together with the nitrogen to which they are attached, form a non-aromatic heterocyclyl optionally substituted with —(CR^{a}R^{b})ₚ-Q-R¹⁰, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR_{c}, —C(O)R^{a}, —OC(O)R^{a}, —C(O)OR^{a}, —SR^{a}, —SO₂R^{a}, —SO₂NR^{a}R^{b}, aryl, and heterocyclyl;
Q is a five- or six-membered heteroaryl, optionally substituted with halogen, methyl, or ethyl;
R¹⁰ is selected from hydrogen, (C₁-C₇)hydrocarbon, (C₁-C₄)haloalkyl, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, and heterocyclyl;
p is 0, 1, 2, 3 or 4;
n is 1, 2, 3 or 4;
R³ and R⁴ are independently selected in each instance from hydrogen, (C₁-C₄)alkyl and cyclopropyl;
R⁵ represents from one to four substituents, selected independently in each instance from hydrogen, (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁-C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, optionally substituted aryl, optionally substituted arylalkyl, optionally substituted (C₃-C₆)cycloalkyl, and optionally substituted heterocyclyl;
R^{a} and R^{b} are independently selected in each instance from hydrogen, (C₁-C₈)hydrocarbon, —OH, and optionally substituted heterocycle; and
R^{c} is (C₁-C₈)hydrocarbon;
wherein said optional substituents are selected in each instance from (C₁-C₄)alkyl, (C₁-C₄)haloalkyl, —OR^{a}, (C₁-C₄)haloalkoxy, (C₁-C₄)alkylthio, —(C₁-C₆)haloalkylthio, halogen, cyano, nitro, (C₁ -C₄)alkylsulfonyl, —NR^{a}R^{b}, —NR^{a}C(O)R^{b}, —C(O)NR^{a}R^{b}, —NR^{a}C(O)OR^{c}, —C(O)R^{a}, —OC(O)R^{a}, —C(O)OR^{a}, —SR^{a}, —SO₂R^{a}, —SO₂NR^{a}R^{b}, aryl, and heterocyclyl.

19. An in vitro method according to claim 18.

20. An in vivo method according to claim 18.
